(19) United States
(12) Patent Application Publication
(10) Pub. No.: US 2011/0275637 A1
(43) Pub. Date: Nov. 10, 2011

(54) PIPERAZINE DERIVATIVES AND THEIR USE AS LEPTIN RECEPTOR MODULATORS

(76) Inventors: Emma Chapman, Cambridge (GB); Michael Higginbottom, Cambridge (GB); Viet-Anh Anne Horgan (nee Nguyen), London (GB); James Horton, Hertfordshire (GB); Iain Simpson, Cambridge (GB); Charles Tyzack, Hampshire (GB)

(21) Appl. No.: 12/996,614

(22) PCT Filed: Jun. 4, 2009
(86) PCT No.: PCT/EP09/56877

§ 371 Date: Jul. 26, 2011

(30) Foreign Application Priority Data
Jun. 4, 2008 (SE) 0801318-7

(51) Int. Cl. A61K 31/5377; C07D 401/12; A61K 31/4439; A61K 31/496; A61K 31/4545; A61P 3/04; A61P 3/10; A61P 5/50; A61P 3/00; A61P 3/06; A61P 1/16; A61P 5/02; A61P 9/12; A61P 7/00; A61P 15/08; A61P 17/00; A61P 15/00; A61P 37/04; A61P 29/00; A61P 9/10; C07D 413/12

(52) U.S. Cl. 514/237.2; 544/131; 546/278.4; 544/360; 546/193; 514/343; 514/253.01; 514/318

(57) ABSTRACT

The present invention relates to new compounds of formula (I), to pharmaceutical compositions comprising these compounds and to the use of these compounds as leptin receptor modulator mimetics in the preparation of medicaments against conditions associated with weight gain, type 2 diabetes and dyslipidemias.

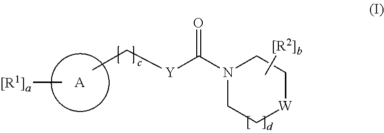

### FIELD OF THE INVENTION

The present application relates to new pyridine derivatives, to pharmaceutical compositions comprising these compounds and to the use of these compounds as leptin receptor modulator mimetics in the preparation of medicaments against conditions associated with weight gain, type 2 diabetes and dyslipidemias.

### BACKGROUND ART

The prevalence of obesity is increasing in the industrialized world. Typically, the first line of treatment is to offer diet and life style advice to patients, such as reducing the fat content of their diet and increasing their physical activity. However, some patients may also need to undergo drug therapy to maintain the beneficial results obtained from adapting the aforementioned diet and lifestyle changes.

Leptin is a hormone synthesized in fat cells that is believed to act in the hypothalamus to reduce food intake and body weight (see, e.g., Bryson, J. M. (2000) Diabetes, Obesity and Metabolism 2: 83-89).

It has been shown that in obese humans the ratio of leptin in the cerebrospinal fluid to that of circulating leptin is decreased (Koistinen et al., (1998) Eur. J. Clin. Invest. 28: 894-897). This suggests that the capacity for leptin transport into the brain is deficient in the obese state. Indeed, in animal models of obesity (NZO mouse and Koletsky rat), defects in leptin transport have been shown to result in reduced brain leptin content (Kastin, A. J. (1999) Peptides 20: 1449-1453; Banks, W. A. et al., (2002) Brain Res. 950: 130-136). In studies involving dietary-induced obese rodents (a rodent model that is believed to more closely resemble human obesity, see, e.g., Van Heek et al. (1997) J. Clin. Invest. 99: 385-390), excess leptin administered peripherally was shown to be ineffective in reducing food intake and body weight, whereas leptin injected directly into the brain was effective in reducing food intake and body weight. It has also been shown that in obese humans with excess circulating leptin, the signaling system became desensitized to the continual stimulation of the leptin receptors (Mantzoros, C. S. (1999) Ann. Intern. Med. 130: 671-680).

Amgen has conducted clinical trials with recombinant methionyl human leptin. The results from these trials were mixed, as even in the presence of high plasma concentrations of leptin weight loss was variable, and the average weight reduction in the cohort of patients tested relatively small (Obesity Strategic Perspective, Datamonitor, 2001).

Several attempts at finding active fragments have been reported in the literature since the discovery of the leptin gene coding sequence. An example is by Samson et al. (1996) Endocrinol. 137: 5182-5185 which describes an active fragment at the N-terminal (22 to 56). This sequence was shown to reduce food intake when injected ICV whereas a sequence taken at the C-terminal was shown not to have any effect. Leptin fragments are also disclosed in International Patent Application WO 97/46585.

Other reports looking at the C-terminus part of the sequence reported a possible stimulation of luteinising hormone production by a 116-130 fragment (Gonzalez et al., (1999) Neuroendocrinology 70:213-220) and an effect on GH production following GHRH administration (fragment 126-140) (Hanew (2003) Eur. J. Endocrin. 149: 407-412).

Leptin has recently been associated with inflammation. It has been reported that circulating leptin levels rise during bacterial infection and in inflammation (see Otero, M et al. (2005) FEBS Lett. 579: 295-301 and references therein). Leptin can also act to increase inflammation by enhancing the release of pro-inflammatory cytokines TNF and IL-6 from inflammatory cells (Zarkesh-Esfahani, H. et al. (2001) J. Immunol. 167: 4593-4599). These agents in turn can contribute to the insulin resistance commonly seen in obese patients by reducing the efficacy of insulin receptor signaling (Lyon, C. J. et al. (2003) Endocrinol. 44: 2195-2200). Continuous low grade inflammation is believed to be associated with obesity (in the presence and absence of insulin resistance and Type II diabetes) (Browning et al. (2004) Metabolism 53: 899-903, Inflammatory markers elevated in blood of obese women; Mangge et al. (2004) Exp. Clin. Endocrinol. Diabetes 112: 378-382, Juvenile obesity correlates with serum inflammatory marker C-reactive protein; Maachi et al. (2004) Int. J. Obes. Relat. Metab. Disord. 28: 993-997, Systemic low grade inflammation in obese people). Leptin has also been implicated in the process of atherogenesis, by promoting lipid uptake into macrophages and endothelial dysfunction, thus promoting the formation of atherosclerotic plaques (see Lyon, C. J. et al. (2003) Endocrinol. 144: 2195-2200).

Leptin has also been shown to promote the formation of new blood vessels (angiogenesis) a process implicated in the growth of adipose tissue (Bouloumie A, et al. (1998) Circ. Res. 83: 1059-1066). Angiogenesis has also been implicated in diabetic retinopathy (Suganami, E. et al. (2004) Diabetes. 53: 2443-2448).

Angiogenesis is also believed to be involved with the growth of new blood vessels that feed abnormal tumour cells. Elevated leptin levels have been associated with a number of cancers, in particular breast, prostate and gastrointestinal cancers in humans (Somasundar P. et al. (2004) J. Surg. Res. 116: 337-349).

Leptin receptor agonists may also be used in the manufacture of a medicament to promote wound healing (Gorden, P. and Gavrilova, O. (2003) Current Opinion in Pharmacology 3: 655-659).

Further, it has been shown that elevating leptin signaling in the brain may represent an approach for the treatment of depressive disorders (Lu, Xin-Yun et al. (2006) PNAS 103: 1593-1598).

### DISCLOSURE OF THE INVENTION

It has surprisingly been found that compounds of formula (I) are effective in reducing body weight and food intake in rodents. While not wishing to be bound by theory, it is proposed that the compounds of formula I modulate the leptin receptor signaling pathway.

In some embodiments, compounds with leptin receptor agonistic like properties can be useful for the treatment of disorders relating to leptin signaling, as well as conditions associated with weight gain, such as obesity. The inventors hypothesized that small molecule CNS penetrant leptin mimetics would be able to by-pass the limiting uptake system into the brain. Further, assuming that this situation mirrors the human obese condition, the inventors believe that a CNS-penetrant leptinoid with a relatively long duration of action would make an effective therapy for the obese state and its attendant complications, in particular (but not limited to) diabetes.

In other embodiments, compounds with leptin receptor antagonistic like properties could be useful for the treatment of inflammation, atherosclerosis, diabetic retinopathy and nephropathy.

In one aspect, the disclosure relates to a compound of formula (I),

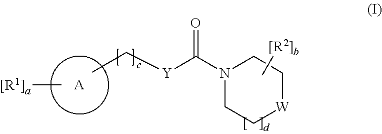

or a pharmaceutically acceptable salt, solvate, hydrate, geometrical isomer, tautomer, optical isomer or N-oxide thereof, wherein:
A is a pyridine ring;

### Y is O, N(R³) or CH₂;

### W is O, N(R⁴) or CH₂;

each R¹ is independently selected from C₁₋₄-alkyl, C₁₋₄-alkoxy, halogen, cyano and CF₃;
each R² is independently selected from hydroxy and C₁₋₄-alkyl;
R³ is hydrogen or C₁₋₄-alkyl;
R⁴ is selected from hydrogen, C₁₋₆-alkyl, C₁₋₆-acyl, phenyl and benzyl, wherein phenyl and benzyl are optionally substituted with one or more substituents selected from halogen, cyano, CF₃, C₁₋₆-alkyl, C₁₋₆-alkoxy, phenyl and phenoxy;
a and b are each independently 0, 1 or 2;
c is 1 or 2; and
d is 0, 1 or 2;
provided that the compound is not selected from the group consisting of:
N-(4-pyridinylmethyl)-4-morpholinecarboxamide;
4-(3-methylphenyl)-N-(2-pyridinylmethyl)-1-piperazinecarboxamide;
4-(4-fluorophenyl)-N-(3-pyridinylmethyl)-1-piperazinecarboxamide;
N-(2-pyridinylmethyl)-4-morpholinecarboxamide;
4-(2-methylphenyl)-N-(3-pyridinylmethyl)-1-piperazinecarboxamide;
4-(5-chloro-2-methylphenyl)-N-(3-pyridinylmethyl)-1-piperazinecarboxamide;
N-(3-pyridinylmethyl)-4-morpholinecarboxamide;
(2R,5S)-4-[4-cyano-3-(trifluoromethyl)phenyl]-N,2,5-trimethyl-N-(2-pyridinylmethyl)-1-piperazinecarboxamide;
(2R,5S)-4-[4-cyano-3-(trifluoromethyl)phenyl]-2,5-dimethyl-N-[(3-methyl-2-pyridinyl)methyl]-1-piperazinecarboxamide;
(2-pyridinyl)methyl 4-methylpiperazine-1-carboxylate;
(2R,5S)-4-[4-cyano-3-(trifluoromethyl)phenyl]-2,5-dimethyl-N-(3-pyridinylmethyl)-1-piperazinecarboxamide;
2-(2-pyridinyl)ethyl 4-methylpiperazine-1-carboxylate;
4-phenyl-N-[2-(2-pyridinyl)ethyl]-1-piperazinecarboxamide;
4-(2,3-dimethylphenyl)-N-(3-pyridinylmethyl)-1-piperazinecarboxamide;
(2R,5S)-4-[4-cyano-3-(trifluoromethyl)phenyl]-2,5-dimethyl-N-[2-(4-pyridinyl)ethyl]-1-piperazinecarboxamide;
4-(4-chlorophenyl)-N-(3-pyridinylmethyl)-1-piperidinecarboxamide;
N-[2-(4-pyridinyl)ethyl]-4-morpholinecarboxamide;
4-phenyl-N-(3-pyridinylmethyl)-1-piperazinecarboxamide;
(2R,5S)-4-[4-cyano-3-(trifluoromethyl)phenyl]-N,2,5-trimethyl-N-(3-pyridinylmethyl)-1-piperazinecarboxamide;
4-phenyl-N-(2-pyridinylmethyl)-1-piperazinecarboxamide;
N-[[3-chloro-5-(trifluoromethyl)-2-pyridinyl]methyl]-4-morpholinecarboxamide;
(2R,5S)—N-[(6-chloro-2-pyridinyl)methyl]-4-[4-cyano-3-(trifluoromethyl)phenyl]-2,5-dimethyl-1-piperazinecarboxamide;
(2R,5S)—N-[(6-chloro-3-pyridinyl)methyl]-4-[4-cyano-3-(trifluoromethyl)phenyl]-2,5-dimethyl-1-piperazinecarboxamide;
1-[3-[3-chloro-5-(trifluoromethyl)-2-pyridinyl]-1-oxopropyl]-4-phenyl-piperazine;
1-[3-(2-pyridyl)propionyl]-piperidine;
1-methyl-4-[1-oxo-3-(3-pyridinyl)propyl]-piperazine;
1-[3-[3-chloro-5-(trifluoromethyl)-2-pyridinyl]-1-oxopropyl]-4-methyl-piperazine;
4-[3-[3-chloro-5-(trifluoromethyl)-2-pyridinyl]-1-oxopropyl]-morpholine;
(6-methylpyridin-2-yl)methyl 4-(2,4,6-trimethoxybenzyl)-piperazine-1-carboxylate;
4-(5-fluoro-2-methoxybenzyl)-N-[2-(pyridin-2-yl)ethyl]-1-piperazinecarboxamide;
N-[(6-methoxy-3-pyridinyl)methyl]-4-phenyl-1-piperazinecarboxamide;
4-(4-fluorophenyl)-N-[(6-methoxy-3-pyridinyl)methyl]-1-piperazinecarboxamide;
4-(2-fluorophenyl)-N-[(6-methoxy-3-pyridinyl)methyl]-1-piperazinecarboxamide;
(2R,5S)-4-[4-cyano-3-(trifluoromethyl)phenyl]-N-[(6-methoxy-3-pyridinyl)methyl]-2,5-dimethyl-1-piperazinecarboxamide;
1-[1-oxo-3-(3-pyridinyl)propyl]-piperazine;
4-[1-oxo-3-(2-pyridinyl)propyl]-morpholine;
4-(3-chlorophenyl)-N-[(6-methoxy-3-pyridinyl)methyl]-1-piperazinecarboxamide;
4-(4-methoxyphenyl)-N-(3-pyridinylmethyl)-1-piperazinecarboxamide; and
N-[(1-oxidopyridin-3-yl)methyl]piperidine-1-carboxamide.

In a preferred embodiment, Y is O or N(R³).

R¹ is preferably C₁₋₄-alkyl, more preferably methyl.

R² is preferably methyl or hydroxy.

When W is N(R⁴), R⁴ is preferably selected from hydrogen, methyl, ethyl, acetyl and phenyl.

d is preferably 0 or 1, and most preferably 1.

Specific preferred compounds according to the disclosure are those selected from the group consisting of:
pyridin-4-ylmethyl morpholine-4-carboxylate;
pyridin-4-ylmethyl (3R)-3-hydroxypyrrolidine-1-carboxylate;
pyridin-4-ylmethyl (2R,6S)-2,6-dimethylmorpholine-4-carboxylate;
pyridin-4-ylmethyl 4-ethylpiperazine-1-carboxylate;
pyridin-4-ylmethyl 4-phenylpiperazine-1-carboxylate;
2-pyridin-4-ylethyl (2R,6S)-2,6-dimethylmorpholine-4-carboxylate;
(2,6-dimethylpyridin-4-yl)methyl morpholine-4-carboxylate;
(2,6-dimethylpyridin-4-yl)methyl (2R,6S)-2,6-dimethylmorpholine-4-carboxylate;
(2,6-dimethylpyridin-4-yl)methyl piperazine-1-carboxylate;
(2,6-dimethylpyridin-4-yl)methyl 4-ethylpiperazine-1-carboxylate;
(2,6-dimethylpyridin-4-yl)methyl (3S)-3-hydroxypiperidine-1-carboxylate;
(2,6-dimethylpyridin-4-yl)methyl 4-methylpiperazine-1-carboxylate;
(2,6-dimethylpyridin-4-yl)methyl (2S)-2,4-dimethylpiperazine-1-carboxylate;
(2,6-dimethylpyridin-4-yl)methyl 4-acetylpiperazine-1-carboxylate;
pyridin-3-ylmethyl (2R,6S)-2,6-dimethylmorpholine-4-carboxylate;
(6-methylpyridin-3-yl)methyl morpholine-4-carboxylate;
(6-methylpyridin-3-yl)methyl 4-ethylpiperazine-1-carboxylate;
(2-methylpyridin-3-yl)methyl morpholine-4-carboxylate;
(6-methylpyridin-2-yl)methyl morpholine-4-carboxylate;
(2,4-dimethylpyridin-3-yl)methyl morpholine-4-carboxylate;
N-ethyl-N-(pyridin-4-ylmethyl)morpholine-4-carboxamide;
N-[(2,6-dimethylpyridin-4-yl)methyl]morpholine-4-carboxamide; and
N-[(2,6-dimethylpyridin-4-yl)methyl]-N-ethylmorpholine-4-carboxamide.

Another aspect of the present disclosure is a compound of formula (I) for use in therapy.

In a further aspect, the invention relates to a compound of formula (I) for use in the treatment or prevention of any of the disorders or conditions described herein.

In a yet further aspect, the invention relates to the use of the compounds of formula (I) in the manufacture of a medicament for the treatment or prevention of any of the disorders or conditions described herein.

In some embodiments, said compounds may be used in the manufacture of a medicament for the treatment or prevention of a condition that is prevented, treated, or ameliorated by selective action via the leptin receptor.

In some embodiments, compounds of formula (I) may be used for the treatment or prevention of conditions (in particular, metabolic conditions) that are associated with weight gain. Conditions associated with weight gain include diseases, disorders, or other conditions that have an increased incidence in obese or overweight subjects. Examples include: lipodystrophy, HIV lipodystrophy, diabetes (type 2), insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain, macular degeneration. In some embodiments, compounds of formula (I) may also be used in the manufacture of a medicament for maintaining weight loss of a subject.

In some embodiments, compounds of formula (I) which are leptin receptor agonist mimetics may also be used to promote wound healing.

In some embodiments, compounds of formula (I) which are leptin receptor agonist mimetics may also be used for the treatment or prevention of conditions that cause a decrease in circulating leptin concentrations, and the consequent malfunction of the immune and reproductive systems. Examples of such conditions and malfunctions include severe weight loss, dysmenorrhea, amenorrhea, female infertility, immunodeficiency and conditions associated with low testosterone levels.

In some embodiments, compounds of formula (I) which are leptin receptor agonist mimetics may also be used for the treatment or prevention of conditions caused as a result of leptin deficiency, or a leptin or leptin receptor mutation.

In some other embodiments, compounds of formula (I) which are leptin receptor antagonist mimetics may be used for the treatment or prevention of inflammatory conditions or is diseases, low level inflammation associated with obesity and excess plasma leptin and in reducing other complications associated with obesity including atherosclerosis, and for the correction of insulin resistance seen in Metabolic Syndrome and diabetes.

In some embodiments, compounds of formula (I) which are leptin receptor antagonist mimetics can be used for the treatment or prevention of inflammation caused by or associated with: cancer (such as leukemias, lymphomas, carcinomas, colon cancer, breast cancer, lung cancer, pancreatic cancer, hepatocellular carcinoma, kidney cancer, melanoma, hepatic, lung, breast, and prostate metastases, etc.); auto-immune disease (such as organ transplant rejection, lupus erythematosus, graft v. host rejection, allograft rejections, multiple sclerosis, rheumatoid arthritis, type I diabetes mellitus including the destruction of pancreatic islets leading to diabetes and the inflammatory consequences of diabetes); autoimmune damage (including multiple sclerosis, Guillam Barre Syndrome, myasthenia gravis); cardiovascular conditions associated with poor tissue perfusion and inflammation (such as atheromas, atherosclerosis, stroke, ischaemia-reperfusion injury, claudication, spinal cord injury, congestive heart failure, vasculitis, haemorrhagic shock, vasospasm following subarachnoid haemorrhage, vasospasm following cerebrovascular accident, pleuritis, pericarditis, the cardiovascular complications of diabetes); ischaemia-reperfusion injury, ischaemia and associated inflammation, restenosis following angioplasty and inflammatory aneurysms; epilepsy, neurodegeneration (including Alzheimer's Disease), arthritis (such as rheumatoid arthritis, osteoarthritis, rheumatoid spondylitis, gouty arthritis), fibrosis (for example of the lung, skin and liver), multiple sclerosis, sepsis, septic shock, encephalitis, infectious arthritis, Jarisch-Herxheimer reaction, shingles, toxic shock, cerebral malaria, Lyme's disease, endotoxic shock, gram negative shock, haemorrhagic shock, hepatitis (arising both from tissue damage or viral infection), deep vein thrombosis, gout; conditions associated with breathing difficulties (e.g. chronic obstructive pulmonary disease, impeded and obstructed airways, bronchoconstriction, pulmonary vasoconstriction, impeded respiration, chronic pulmonary inflammatory disease, silicosis, pulmonary sarcosis, cystic fibrosis, pulmonary hypertension, pulmonary vasoconstriction, emphysema, bronchial allergy and/or inflammation, asthma, hay fever, rhinitis, vernal conjunctivitis and adult respiratory distress syndrome); conditions associated with inflammation of the skin (including psoriasis, eczema, ulcers, contact dermatitis); conditions associated with inflammation of is the bowel (including Crohn's disease, ulcerative colitis and pyresis, irritable bowel syndrome, inflammatory bowel disease); HIV (particularly HIV infection), cerebral malaria, bacterial meningitis, osteoporosis and other bone resorption diseases, osteoarthritis, infertility from endometriosis, fever and myalgia due to infection, and other conditions mediated by excessive anti-inflammatory cell (including neutrophil, eosinophil, macrophage and T-cell) activity.

In some embodiments, compounds of formula (I) which are leptin receptor antagonists mimetics may be used for the treatment or prevention of macro or micro vascular complications of type 1 or 2 diabetes, retinopathy, nephropathy, autonomic neuropathy, or blood vessel damage caused by ischaemia or atherosclerosis.

In some embodiments, compounds of formula (I) which are leptin receptor antagonist mimetics may be used to inhibit angiogenesis. Compounds that inhibit angiogenesis may be used for the treatment or prevention of obesity or complications associated with obesity. Compounds that inhibit angiogenesis may be used for the treatment or prevention of complications associated with inflammation diabetic retinopathy, or tumour growth particularly in breast, prostate or gastrointestinal cancer.

In a further aspect, the disclosure relates to a method for the treatment or prevention of any of the disorders or conditions described herein, which includes administering to a subject (e.g., a subject in need thereof, e.g., a mammal) an effective amount of a compound of formula I.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker is or indicator of suitability for such treatment.

In one embodiment, the disclosure provides a method of monitoring treatment progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In certain method embodiments, a level of Marker or Marker activity in a subject is determined at least once. Comparison of Marker levels, e.g., to another measurement of Marker level obtained previously or subsequently from the same patient, another patient, or a normal subject, may be useful in determining whether therapy according to the disclosure is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radio labeling/assay techniques, blotting/chemiluminescence methods, real-time PCR, and the like.

In some embodiments, it may be advantageous if a compound of formula (I) is able to penetrate the central nervous system. In other embodiments, it may be advantageous if a is compound of formula (I) is not able to penetrate the CNS. In general, it is expected that compounds that are leptin receptor agonist mimetics may be particularly useful for the treatment or prevention of obesity, insulin resistance, or diabetes (particularly glucose intolerance) if these compounds can penetrate the CNS. A person of ordinary skill in the art can readily determine whether a compound can penetrate the CNS. A suitable method that may be used is described in the Biological Methods section.

A leptin receptor response may be measured in any suitable way. In vitro, this may be done be measuring leptin receptor signaling. For example, phosphorylation of Akt, STAT3, STAT5, MAPK, shp2 or the leptin receptor in response to binding of leptin or a compound of the invention to the leptin receptor may be measured. The extent of phosphorylation of Akt, STAT3, STAT5, MAPK, shp2 or the leptin receptor may be determined for example by Western blotting or by ELISA. Alternatively, a STAT reporter assay may be used, for example STAT driven luciferase expression. A cell line expressing the leptin receptor may be used for such assays. In vivo, leptin receptor response may be measured by determining the reduction in food intake and body weight after administration of leptin or a compound of formula (I).

The Biological Methods below describe assays and methods that can be used to determine whether a compound of formula (I) is a leptin receptor agonist mimetic or a leptin receptor antagonist mimetic.

A compound of formula (I) may be administered with or without other therapeutic agents. For example, where it is desired to reduce inflammation, a compound may be administered with an anti-inflammatory agent (for example, disease modifying anti-rheumatic drugs such as methotrexate, sulphasalazine and cytokine inactivating agents, steroids, NSAIDs, cannabinoids, tachykinin modulators, or bradykinin modulators). Where it is desired to provide an anti-tumour effect, a compound may be administered with a cytotoxic agent (for example, methotrexate, cyclophosphamide) or another anti-tumour drug.

Compounds of formula (I) may be radio labeled (for example with tritium or radioactive iodine) for in vitro or in vivo applications, such as receptor displacement studies or receptor imaging.

### DEFINITIONS

The following definitions shall apply throughout the specification and the appended claims.

Unless otherwise stated or indicated, the term “C₁₋₆-alkyl” denotes a straight or branched alkyl group having from 1 to 6 carbon atoms. Examples of said C₁₋₆-alkyl include methyl, ethyl, n-propyl, iso-propyl, n-butyl, iso-butyl, sec-butyl, t-butyl, and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc.

Unless otherwise stated or indicated, the term “C₁₋₆-acyl” denotes a carbonyl group that is attached through its carbon atom to a hydrogen atom (i.e., a formyl group) or to a straight or branched C₁₋₅-alkyl group, where alkyl is defined as above. Examples of said C₁₋₆-acyl include formyl, acetyl, propionyl, n-butyryl, 2-methylpropionyl and n-pentoyl. For parts of the range “C₁₋₆-acyl” all subgroups thereof are contemplated such as C₁₋₅-acyl, C₁₋₄-acyl, C₁₋₃-acyl, C₁₋₂-acyl, C₂₋₆-acyl, C₂₋₅-acyl, C₂₋₄-acyl, C₂₋₃-acyl, C₃₋₆-acyl, C₄₋₅-acyl, etc.

Unless otherwise stated or indicated, the term “C₁₋₆-alkoxy” denotes a straight or branched alkoxy group having from 1 to 6 carbon atoms. Examples of said C₁₋₆-alkoxy include methoxy, ethoxy, n-propoxy, iso-propoxy, n-butoxy, iso-butoxy, sec-butoxy, t-butoxy, and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

“Halogen” refers to fluorine, chlorine, bromine or iodine.

“Hydroxy” refers to the —OH radical.

“Cyano” refers to the —CN radical.

“Optional” or “optionally” means that the subsequently described event or circumstance may but need not occur, and that the description includes instances where the event or circumstance occurs and instances in which it does not.

The term “mammal” includes organisms, which include mice, rats, cows, sheep, pigs, rabbits, goats, and horses, monkeys, dogs, cats, and preferably humans. The subject may be is a human subject or a non human animal, particularly a domesticated animal, such as a dog.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect).

“Prodrugs” refers to compounds that may be converted under physiological conditions or by solvolysis to a biologically active compound of formula (I). A prodrug may be inactive when administered to a subject in need thereof, but is converted in vivo to an active compound of formula (I). Prodrugs are typically rapidly transformed in vivo to yield the parent compound, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2^{nd} Ed., Elsevier Academic Press (2004), pp. 498-549). Prodrugs may be prepared by modifying functional groups, such as a hydroxy, amino or mercapto groups, present in a compound of formula (I) in such a way that the modifications are cleaved, either in routine manipulation or in vivo, to the parent compound. Examples of prodrugs include, but are not limited to, acetate, formate and succinate derivatives of hydroxy functional groups or phenyl carbamate derivatives of amino functional groups.

Throughout the specification and the appended claims, a given chemical formula or name shall also encompass all salts, hydrates, solvates, N-oxides and prodrug forms thereof. Further, a given chemical formula or name shall encompass all tautomeric and stereoisomeric forms thereof. Stereoisomers include enantiomers and diastereomers. Enantiomers can be present in their pure forms, or as racemic (equal) or unequal mixtures of two enantiomers. Diastereomers can be present in their pure forms, or as mixtures of diastereomers. Diastereomers also include geometrical isomers, which can be present in is their pure cis or trans forms or as mixtures of those.

The compounds of formula (I) may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

### Compositions

For clinical use, the compounds of formula (I) are formulated into pharmaceutical formulations for various modes of administration. It will be appreciated that the compounds may be administered together with a physiologically acceptable carrier, excipient, or diluent. The pharmaceutical compositions may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration.

Other formulations may conveniently be presented in unit dosage form, e.g., tablets and sustained release capsules, and in liposomes, and may be prepared by any methods well is known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glyco late, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, the compounds may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

### Preparation of Compounds of the Invention

The compounds of formula (I) above may be prepared by, or in analogy with, conventional methods. Formation of the central urethane or urea linker is the key synthetic step in preparing the compounds of formula (I). A large number of activating reagents can be used for the formation of a urethane or urea linker e.g. phosgene to form chloroformate of alcohols, or carbonyldiimidazole (CDI) to form imidazole carboxylates. Typically the urethane linkers incorporated into compounds of formula (I) have been synthesized utilizing bis-(4-nitrophenyl)carbonate as the activating agent. The preparation of intermediates and compounds according to the examples of the present invention may in particular be illuminated by the following Schemes 1 and 2. Definitions of variables in the structures in the schemes herein are commensurate with those of corresponding positions in the formulae delineated herein.

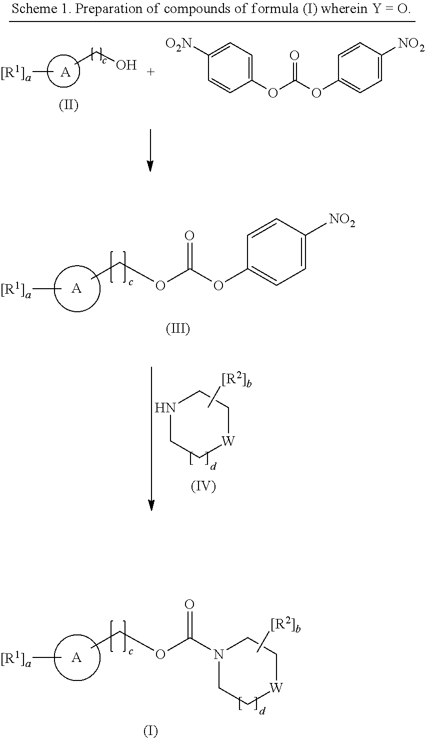

wherein A, W, R¹, R² and a-d are as defined in formula (I).

Compounds of formula (I) wherein Y═O can easily be prepared in only a few steps. In the first step, an alcohol derivative of formula (II) is activated with bis-(4-nitrophenyl)-carbonate in the presence of a base (such as NMM) in an aprotic solvent (such as DCM) to give the corresponding carbonate of formula (III). The carbonate intermediate (III) is then subsequently treated with the appropriate N-heterocycle of formula (IV) in the presence of a base (such as DIPEA or NEt₃) and optionally an activating agent (such as DMAP) in an aprotic solvent (such as DMF), which results in the formation of the desired compound of formula (I).

The formation of the urethane is typically a two step process but this may also be performed in a one-pot reaction by formation of the activated intermediate in situ.

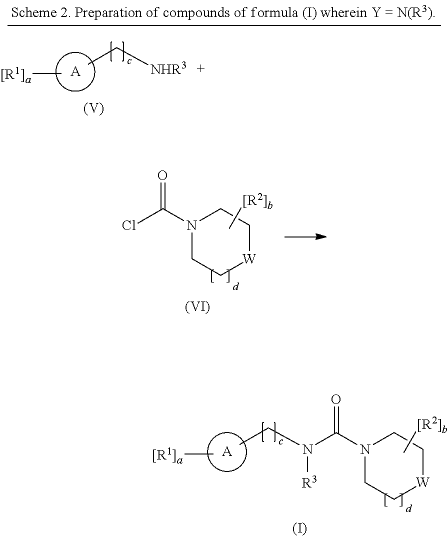

wherein A, W, R¹-R³ and a-d are as defined in formula (I).

Compounds of formula (I) wherein Y═N(R³) are easily prepared by condensation of an amino derivative of formula (V) with the appropriate N-heterocyclylcarbonyl chloride derivative of formula (VI) in the presence of a base (such as DIPEA) or an activating agent (such as DMAP) in an aprotic solvent (such as DMF or DCM).

The necessary starting materials for preparing the compounds of formula (I) are either commercially available, or may be prepared by methods known in the art.

The processes described below in the experimental section may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. Examples of protecting groups are t-butoxycarbonyl (Boc), benzyl and trityl (triphenylmethyl). The methods described above may also additionally include steps, either before or after the steps described specifically herein, to add or remove suitable protecting is groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The following abbreviations have been used:

| | |
|---|---|
| aq | Aqueous |
| Boc | tert-Butoxy carbonyl |
| cat. | Catalytic amount |
| DCM | Dichloromethane |
| DEAD | Diethyl azodicarboxylate |
| DIPEA | N, N-Diisopropylethylamine |
| DMAP | N,N-Dimethylaminopyridine |
| DMF | N, N-Dimethylformamide |
| ES⁺ | Electrospray |
| Et₂O | Diethyl ether |
| EtOAc | Ethyl acetate |
| HIV | Human immunodeficiency virus |
| HPLC | High performance liquid chromatography |
| ICV | Intracerebroventricular |
| LCMS | Liquid Chromatography Mass Spectrometry |
| M | Molar |
| [MH]⁺ | Protonated molecular ion |
| NEt₃ | Triethylamine |
| NMM | N-Methyl morpholine |
| RP | Reverse Phase |
| r.t. | Room temperature |
| sat | Saturated |
| tert | Tertiary |
| TFA | Trifluoroacetic acid |
| THF | Tetrahydrofuran |

Embodiments of the disclosure are described in the following examples with reference to the accompanying drawings, in which:

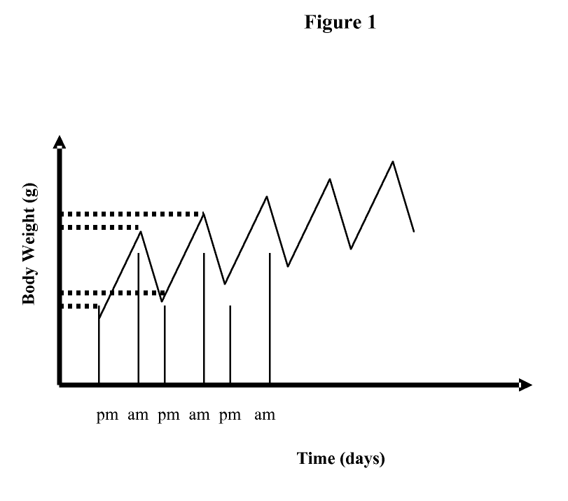

FIG. 1 is a schematic drawing illustrating weight gain and weight loss in mice during dark and light phases, respectively. The graph illustrates the large nocturnal weight increase versus the comparatively small body weight change over 24 hours

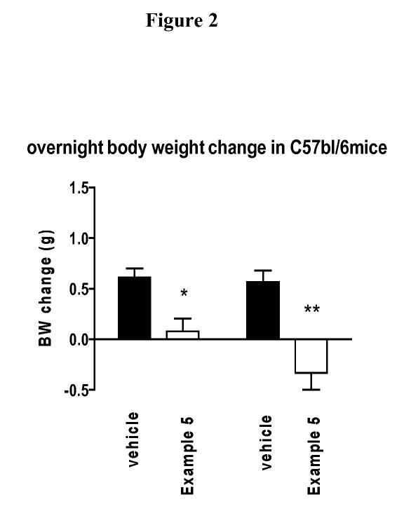

FIG. 2 shows the effect of Example 5 on the body weight in mice between the beginning of the dark phase and the beginning of the light phase (pm-am).

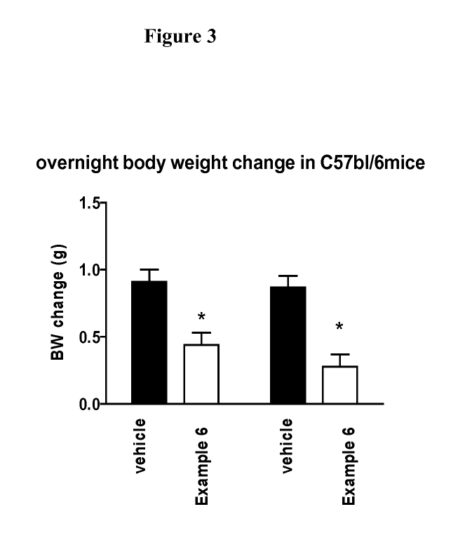

FIG. 3 shows the effect of Example 6 on the body weight in mice between the beginning of the dark phase and the beginning of the light phase (pm-am).

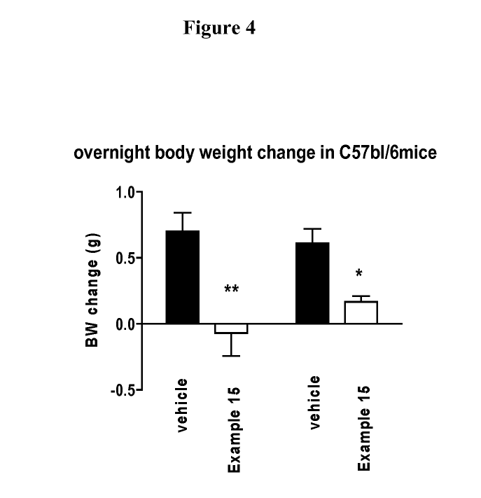

FIG. 4 shows the effect of Example 15 on the body weight in mice between the beginning of the dark phase and the beginning of the light phase (pm-am).

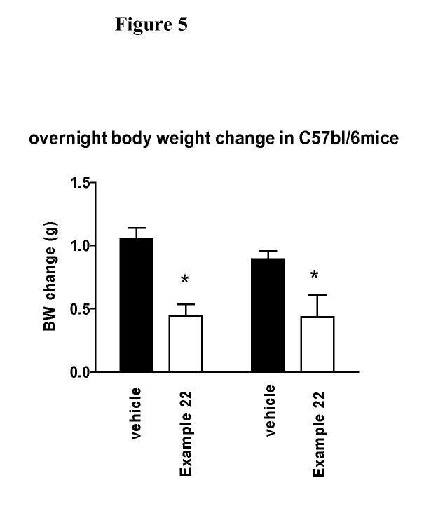

FIG. 5 shows the effect of Example 22 on the body weight in mice between the beginning of the dark phase and the beginning of the light phase (pm-am).

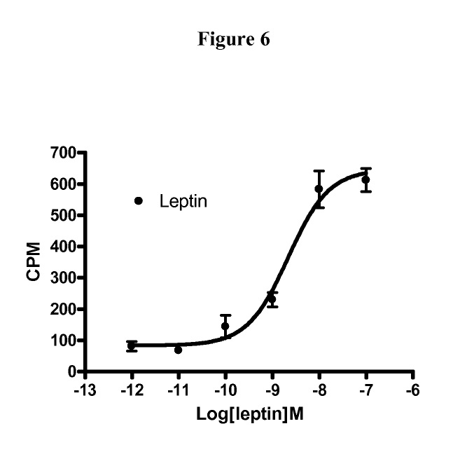

FIG. 6 shows the concentration-dependent increase in [³H]-thymidine incorporation by JEG-3 cells for leptin

The recitation of a listing of chemical groups in any definition of a variable herein includes definitions of that variable as any single group or combination of listed groups. The recitation of an embodiment herein includes that embodiment as any single embodiment or in combination with any other embodiments or portions thereof.

The disclosure will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All references and publications cited herein are hereby incorporated by reference in their entirety.

### EXAMPLES AND INTERMEDIATE COMPOUNDS

### Experimental Methods

All reagents were commercial grade and were used as received without further purification, unless otherwise specified. Commercially available anhydrous solvents were used for reactions conducted under inert atmosphere. Reagent grade solvents were used in all other cases, unless otherwise specified. Analytical LCMS was performed on a Waters ZQ mass spectrometer connected to an Agilent 1100 HPLC system. Analytical HPLC was performed on an Agilent 1100 system. High-resolution mass spectra (HRMS) were obtained on an Agilent MSD-TOF connected to an Agilent 1100 HPLC system. During the analyses the calibration was checked by two masses and automatically corrected when needed. Spectra are acquired in positive electrospray mode. The acquired mass range was m/z 100-1100. Profile detection of the mass peaks was used. Normal phase is chromatography was performed on a Flash Master Personal system equipped with 20 g Strata SI-1 silica gigatubes. Reverse phase chromatography was performed on a Gilson system equipped with Merck LiChoprep® RP-18 (40-63 μm) 460×26 mm column, 30 mL/min, gradient of methanol in water from 0% to 100%. Preparative HPLC was performed on a Gilson system equipped with Phenomenex Hydro RP 150×20 mm, 20 mL/min, gradient of acetonitrile in water. The compounds were automatically named using ACD 6.0.

Analytical HPLC and LCMS data were obtained with:

System A: Phenomenex Synergi Hydro RP (30×4.6 mm, 4 μm), gradient 5-100% CH₃CN (+0.085% TFA) in H₂O (+0.1% TFA), 1.5 mL/min, gradient time 1.75 min, 200-300 nm, 30° C.;
System B: Phenomenex Synergi Hydro RP (150×4.6 mm, 4 μm), gradient 5-100% CH₃CN (+0.085% TFA) in H₂O (+0.1% TFA), 1.5 mL/min, gradient time 7 min, 200-300 nm, 30° C.;
System C: Phenomenex Synergi Hydro RP (150×4.6 mm, 4 μm), gradient 0-20% CH₃CN (+0.085% TFA) in H₂O (+0.1% TFA), 1.5 mL/min, gradient time 7 min, 200-300 nm, 30° C.;
System D: Phenomenex Synergi Hydro RP (150×4.6 mm, 4 μm), gradient 5-100% CH₃CN in H₂O (+0.1% HCO₂H), 1.0 mL/min, gradient time 8 min, 30° C.;
System E: Phenomenex Synergi Hydro RP (150×4.6 mm, 4 μm), gradient 0-20% CH₃CN (+0.085% TFA) in H₂O (+0.1% TFA), 1.0 mL/min, gradient time 8 min, 30° C.;
System F: Phenomenex Synergi Hydro RP (150×4.6 mm, 4 μm), gradient 5-100% CH₃CN (+0.085% TFA) in H₂O (+0.1% TFA), 1.0 mL/min, gradient time 8 min, 30° C.; or
System G: Phenomenex Synergi Hydro RP (150×4.6 mm, 4 μm), gradient 0-50% CH₃CN (+0.085% TFA) in H₂O (+0.1% TFA), 1.5 mL/min, gradient time 7 min, 200-300 nm, 30° C.

### Intermediate 1

### 4-Nitrophenyl (pyridin-4-yl)methyl carbonate

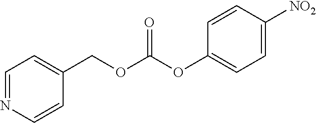

The title compound was prepared according to a literature procedure described by Veber et is al, J. Org. Chem. 1977, 42, 3286-3288.

### Intermediate 2

### (2,6-Dimethylpyridin-4-yl)methyl 4-nitrophenyl carbonate

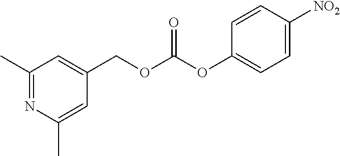

A suspension of (2,6-dimethyl-pyridin-4-yl)-methanol (9.14 g, 66.7 mmol; prepared according to a procedure described by Katz et. al, Synthetic Communications 1989, 19, 317-325) in DCM (40 mL) was added to a solution of bis-(4-nitrophenyl)carbonate (20.3 g, 66.7 mmol) in DCM (200 mL), followed by NMM (7.34 mL). The reaction mixture was stirred overnight, washed with sat aq NaHCO₃ solution (5×100 mL), dried (MgSO₄) and concentrated in vacuo to give an orange solid. This solid was recrystallised from EtOAc (˜25 mL) to give (2,6-dimethylpyridin-4-yl)methyl 4-nitrophenyl carbonate (11.52 g) as an off white solid. The resulting filtrate was concentrated and the residue obtained was further recrystallised from EtOAc (15 mL) with a drop of heptane to give another 4.76 g of the product as an off white solid. Combined yield 16.28 g (81%).

### Intermediate 3

### 4-Nitrophenyl (pyridin-3-yl)methyl carbonate

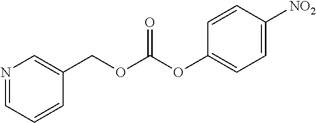

To a solution of 3-pyridylmethanol (0.80 g, 7.34 mmol) in DCM (40 mL) was added bis-(4-nitrophenyl)carbonate (2.23 g, 7.34 mmol) followed by NMM (0.81 mL, 7.34 mmol). The reaction mixture was stirred at r.t. for 66 hours, washed with sat aq NaHCO₃ solution (6×20 mL), dried (MgSO₄) and concentrated in vacuo to give 4-nitrophenyl (pyridin-3-yl)methyl carbonate (1.67 g, 55% yield) as an orange solid.

### Intermediate 4

### (6-Methylpyridin-3-yl)methyl 4-nitrophenyl carbonate

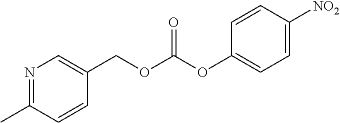

To a solution of methyl 6-methylnicotinate (5.0 g, 33 mmol) in THF (120 mL) under argon at −78° C. was added diisobutylaluminium hydride (1M in hexane, 66 mL, 66 mmol). The reaction mixture was allowed to warm to r.t. and stirred for 4 days. DCM (120 mL) was added. The mixture was added to a saturated aqueous solution of Rochelle salt (150 mL) at 0° C. The mixture was stirred until two layers had formed. The phases were separated and the aqueous phase was extracted with DCM (2×150 mL). The combined organic phases were dried (MgSO₄) and concentrated in vacuo to give (6-methyl-pyridin-3-yl)-methanol as a yellow oil, which was used without further purification in the next step.

Analytical LCMS: purity 83% (System A, R_{T}=0.44 min), ES⁺: 123.9 [MH]⁺.

To a solution of the (6-methyl-pyridin-3-yl)-methanol in DCM (180 mL) was added bis-(4-nitrophenyl)carbonate (10.4 g, 34 mmol) followed by NMM (3.75 mL, 34 mmol). The reaction mixture was stirred at r.t. for 42 hours and then concentrated in vacuo. The residue was dissolved in DCM (100 mL), washed with sat aq NaHCO₃ solution (6×100 mL), dried (MgSO₄) and concentrated in vacuo. The crude product was recrystallised from EtOAc to give (6-methylpyridin-3-yl)methyl 4-nitrophenyl carbonate (7.38 g, 77%) as an orange solid.

Analytical LCMS: (System A, R_{T}=1.74 min), ES⁺: 289.4 [MH]⁺.

### Intermediate 5

### (2-Methylpyridin-3-yl)methyl 4-nitrophenyl carbonate

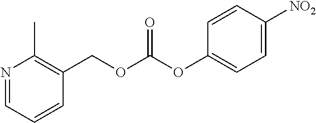

To a solution of methyl 2-methylnicotinate (5.9 g, 39 mmol) in DCM (100 mL) under argon at −78° C. was added diisobutylaluminium hydride (1M in hexane, 97 mL, 97 mmol). The reaction mixture was stirred for 4 hours and then added to a saturated aqueous solution of Rochelle salt (200 mL) at 0° C. The mixture was stirred until two layers had formed. The phases were separated and the aqueous phase was extracted with DCM (2×150 mL). The combined organic phases were dried (MgSO₄) and concentrated in vacuo to give (2-methyl-pyridin-3-yl)-methanol (3.64 g, 76%) as a yellow oil.

Analytical LCMS: (System A, R_{T}=0.44 min), ES⁺: 123.9 [MH]⁺.

To a solution of (2-methyl-pyridin-3-yl)-methanol (3.64 g, 29.6 mmol) in DCM (150 mL) was added bis-4-nitrophenylcarbonate (9.0 g, 29.6 mmol) followed by NMM (3.2 mL, 29.6 mmol). The reaction mixture was stirred at r.t. for 68 hours, washed with sat aq NaHCO₃ solution (6×100 mL), dried (MgSO₄) and concentrated in vacuo to give (2-methylpyridin-3-yl)methyl 4-nitrophenyl carbonate (8.39 g, 98%) as a yellow solid.

Analytical LCMS: (System A, R_{T}=1.74 min), ES⁺: 289.4 [MH]⁺.

### Intermediate 6

### (2,4-Dimethylpyridin-3-yl)methyl 4-nitrophenyl carbonate

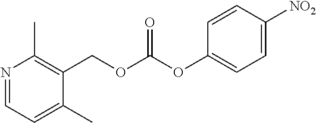

To a solution of ethyl 2,4-dimethylpyridine-3-carboxylate (5.35 g, 30 mmol) in DCM (150 mL) under argon at −78° C. was added diisobutylaluminium hydride (1M in hexane, 100 mL, 100 mmol). The reaction mixture was stirred for 24 hours and then added to a saturated aqueous solution of Rochelle salt (200 mL) at 0° C. The mixture was stirred until two layers had formed. The phases were separated and the aqueous phase was extracted with DCM (2×150 mL). The combined organic phases were dried (MgSO₄) and concentrated in vacuo. The residue was recrystallised from EtOAc to give (2,4-dimethyl-pyridin-3-yl)-methanol (2.76 g, 67%) as a pale yellow solid.

Analytical LCMS: (System A, R_{T}=0.46 min), ES⁺: 138.0 [MH]⁺.

To a solution of (2,4-dimethyl-pyridin-3-yl)-methanol (2.44 g, 17.8 mmol) in DCM (100 mL) was added bis-(4-nitrophenyl)carbonate (5.41 g, 17.8 mmol) followed by NMM (1.95 mL, 17.8 mmol). The reaction mixture was stirred at r.t. for 66 hours, washed with sat aq NaHCO₃ solution (6×100 mL), dried (MgSO₄) and concentrated in vacuo to give a red oil. The oil solidified upon standing and was recrystallised from EtOAc to give (2,4-dimethylpyridin-3-yl)methyl 4-nitrophenyl carbonate (3.36 g, 63%) as a red solid.

Analytical LCMS: (System A, R_{T}=1.82 min), ES⁺: 303.5 [MH]⁺.

### Intermediate 7

### (2,6-Dimethylpyridin-4-yl)methanamine dihydrochloride

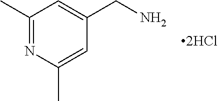

To a solution of 4-(hydroxymethyl)-2,6-dimethylpyridine (10.1 g, 73.6 mmol), phthalimide (11.9 g, 80.9 mmol) and triphenylphosphine (21.2 g, 80.9 mmol) in THF (100 mL) was added DEAD (15.4 g, 88.3 mmol) over 5 minutes. The reaction mixture was stirred for 60 hours. EtOAc (300 mL) and 1M aq HCl solution (100 mL) were added and the organic layer was extracted with 1M aq HCl solution (3×100 mL). The acidic aqueous layers were combined and basified to pH 7.5-8. A precipitate was collected by filtration and dried in vacuo to give 2-((2,6-dimethylpyridin-4-yl)methyl)isoindoline-1,3-dione (17.23 g, 88%) as a white powder.

Analytical HPLC: purity 99% (System G, R_{T}=3.86 min).

2-((2,6-dimethylpyridin-4-yl)methyl)isoindoline-1,3-dione (17.55 g, 65.9 mmol) was added to a mixture of glacial acetic acid (80 mL), concentrated HCl (80 mL) and water (80 mL). The reaction mixture was heated at reflux for 96 hours and then cooled slowly to r.t. A suspension was removed by filtration and the filtrate concentrated in vacuo. The residue was dissolved in water (200 mL), washed with EtOAc (3×100 mL) and the aqueous layer concentrated in vacuo to give (2,6-dimethylpyridin-4-yl)methanamine hydrochloride (5.4 g, 39%) as a white powder.

Analytical HPLC: purity 99.7% (System G, R_{T}=1.28 min); Analytical LCMS: (System A, R_{T}=0.46 min), ES⁺: 137 [MH]⁺.

### Intermediate 8

### (4-Methyl-N-ethylamine)-2,6-dimethylpyridine

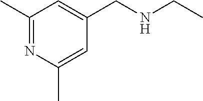

To a solution of 2,6-dimethylpyridylmethyl alcohol (5.0 g, 36.4 mmol) and NEt₃ (12.7 mL, is 91.1 mmol) in DCM (100 mL) was added methanesulfonyl chloride (5.62 mL, 72.9 mmol). The mixture was stirred for one hour at r.t. and then concentrated in vacuo. The residue was dissolved in EtOAc (100 mL), washed with sat aq NaHCO₃ solution (50 mL), brine (50 mL), dried (MgSO₄) and concentrated in vacuo to give a mixture of (2,6-dimethylpyridin-4-yl)methyl methanesulfonate and 4-(chloromethyl)-2,6-dimethylpyridine (4.86 g). A portion (2.0 g) of this mixture was dissolved in DMF (4 mL), N-ethylamine (1.79 mL, 32.2 mmol) added and the mixture heated in a Biotage Initiator microwave at 170° C. for 20 minutes at normal absorption. The reaction mixture was concentrated in vacuo and the residue purified by reverse phase chromatography to give (4-methyl-N-ethylamine)-2,6-dimethylpyridine (702 mg, 28%) as a colourless oil.

### Example 1

### Pyridin-4-ylmethyl morpholine-4-carboxylate hydrochloride

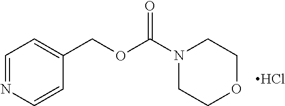

To a solution of Intermediate 1 (274 mg, 1.00 mmol) in DMF (5 mL) was added DIPEA (0.35 mL, 2.00 mmol) and morpholine (91.8 μL, 1.05 mmol) followed by DMAP (30 mg, cat.). The reaction mixture was stirred at r.t. overnight and then concentrated in vacuo. The residue was purified by normal phase chromatography (gradient eluting with MeOH in DCM from 0% to 5%). The residue obtained was dissolved in MeOH (1.0 mL) and 2M

HCl in Et₂O (0.50 mL, 1.00 mmol) was added. The solution was stirred for 10 minutes and concentrated in vacuo to give pyridin-4-ylmethyl morpholine-4-carboxylate hydrochloride (187 mg, 72%) as a white solid.

Analytical HPLC: purity 100% (System B, R_{T}=2.84 min); Analytical LCMS: purity 100% (System D, R_{T}=3.44 min), ES⁺: 222.9 [MH]⁺; HRMS calcd for C₁₁H₁₄N₂O₃: 222.1004, found 222.1008.

### Example 2

### Pyridin-4-ylmethyl (3R)-3-hydroxypyrrolidine-1-carboxylate

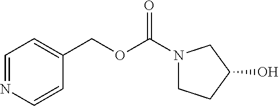

(R)-3-Hydroxypyrrolidine (87 mg, 1 mmol), Intermediate 1 (274 mg, 1.0 mmol), DIPEA (354 μL, 2.0 mmol) and DMAP (10 mg, cat.) were dissolved in DMF (5 mL). The reaction mixture was stirred at r.t. for 16 hours and then concentrated in vacuo. The crude product was purified by normal phase chromatography (gradient eluting with methanol in DCM from 0% to 5%) and then by preparative HPLC (gradient eluting with acetonitrile in water from 5% to 95%) to give pyridin-4-ylmethyl (3R)-3-hydroxypyrrolidine-1-carboxylate (65 mg, 29%) as a colourless oil.

Analytical HPLC: purity 100% (System B, R_{T}=2.63 min); Analytical LCMS: purity 100% (System D, R_{T}=3.21 min), ES⁺: 222.8 [MH]⁺.

### Example 3

### Pyridin-4-ylmethyl (2R,6S)-2,6-dimethylmorpholine-4-carboxylate hydrochloride

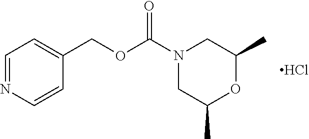

To a solution of Intermediate 1 (1.02 g, 3.72 mmol) in DMF (6 mL) was added DIPEA (0.8 mL, 4.6 mmol), DMAP (10 mg, cat.) and cis-2,6-dimethylmorpholine (0.5 mL, 4.1 mmol). The reaction mixture was stirred at r.t. for 7 days and then concentrated in vacuo. The residue was dissolved in EtOAc (50 mL), washed with 1M aq Na₂CO₃ solution (4×50 mL), dried (MgSO₄) and concentrated in vacuo. The residue was purified by normal phase chromatography (gradient eluting with methanol in DCM from 0 to 5%) to give a white solid. This solid was dissolved in Et₂O and excess 2M HCl in Et₂O was added. The resulting precipitate was collected by filtration, washed with Et₂O and dried in vacuo to give pyridin-4-ylmethyl (2R,6S)-2,6-dimethylmorpholine-4-carboxylate hydrochloride (782 mg, 73%) as a white powder.

Analytical HPLC: purity 99.2% (System B, R_{T}=3.30 min); Analytical LCMS: purity 100% (System D, R_{T}=3.30 min), ES⁺: 251.4 [MH]⁺; HRMS calcd for C₁₃H₁₈N₂O₃: 250.1317, found 250.1322.

### Example 4

### Pyridin-4-ylmethyl 4-ethylpiperazine-1-carboxylate formate

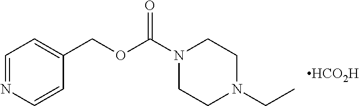

To a solution of Intermediate 1 (1.25 g, 4.67 mmol) in DMF (25 mL) was added DIPEA (1 mL, 5.7 mmol), DMAP (20 mg, cat.) and 1-ethylpiperazine (0.7 mL, 5.5 mmol). The reaction mixture was stirred at r.t. for 48 hours and concentrated in vacuo. The residue was dissolved in EtOAc (50 mL), washed with 1M aq Na₂CO₃ solution (4×50 mL), dried (MgSO₄) and concentrated in vacuo. The residue was purified by reverse phase chromatography (gradient eluting with MeOH in water, with 1% formic acid in each solvent) to give pyridin-4-ylmethyl 4-ethylpiperazine-1-carboxylate formate (358 mg, 26%) as a colourless oil.

Analytical HPLC: purity 99.2% (System C, R_{T}=5.61 min); Analytical LCMS: purity 100% (System E, R_{T}=5.72 min), ES⁺: 250.4 [MH]⁺; HRMS calcd for C₁₃H₁₉N₃O₂: 249.1477, found 249.1484.

### Example 5

### Pyridin-4-ylmethyl 4-phenylpiperazine-1-carboxylate dihydrochloride

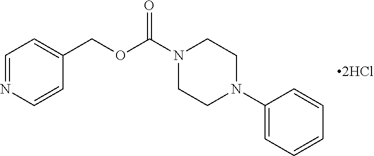

To a solution of Intermediate 1 (1.63 g, 5.95 mmol) in DMF (20 mL) was added triethylamine (1.0 mL, 7.2 mmol), DMAP (20 mg, cat.) and 1-phenylpiperazine (0.91 mL, 5.95 mmol). The reaction mixture was stirred at r.t. for 7 days and then concentrated in vacuo. The residue was dissolved in EtOAc (50 mL), washed with 1M aq Na₂CO₃ solution (4×50 mL), dried (MgSO₄) and concentrated in vacuo. The residue was purified by normal phase chromatography (gradient eluting with MeOH in DCM from 0% to 2%) followed by reverse phase chromatography. The product was dissolved in DCM (10 mL), treated with 2M HCl in Et₂O (5 mL, 10 mmol) and concentrated in vacuo to give pyridin-4-ylmethyl 4-phenylpiperazine-1-carboxylate dihydrochloride (621 mg, 28%) as a light pink solid.

Analytical HPLC: purity 99.5% (System B, R_{T}=3.76 min); Analytical LCMS: purity 100% (System F, R_{T}=5.52 min), ES⁺: 298.4 [MH]⁺; HRMS calcd for C₁₇H₁₉N₃O₂: 297.1477, found 297.1485.

### Example 6

### 2-Pyridin-4-ylethyl (2R,6S)-2,6-dimethylmorpholine-4-carboxylate

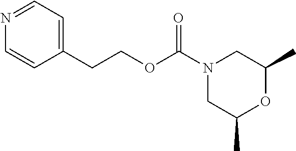

To a solution of 4-(2-hydroxyethyl)pyridine (0.25 mL, 2.2 mmol) in DCM (10 mL) was added bis-(p-nitrophenyl)carbonate (0.67 g, 2.2 mmol) and NMM (0.24 mL, 2.2 mmol). The reaction mixture was stirred at r.t. for 2 days and then concentrated in vacuo to give an orange oil. The oil was dissolved in EtOAc (30 mL), washed with sat aq NaHCO₃ solution (3×20 mL), dried (MgSO₄) and concentrated in vacuo to give 4-nitrophenyl 2-(pyridin-4-yl)ethyl carbonate as an oil which was used without further purification in the next step.

To a solution of 4-nitrophenyl 2-(pyridin-4-yl)ethyl carbonate (˜2.2 mmol) in DMF (10 mL) was added DIPEA (0.383 mL, 2.2 mmol) and cis-2,6-dimethylmorpholine (0.27 mL, 2.2 mmol). The reaction mixture was stirred overnight at r.t. and then concentrated in vacuo. The residue was purified by normal phase chromatography (gradient eluting with MeOH in DCM from 0% to 2%) followed by reverse phase chromatography to give 2-pyridin-4-ylethyl (2R,6S)-2,6-dimethylmorpholine-4-carboxylate (128 mg, 22%) as a light yellow oil.

Analytical HPLC: purity 99.6% (System B, R_{T}=3.44 min); Analytical LCMS: purity 100% (System F, R_{T}=5.18 min), ES⁺: 265.6 [MH]⁺; HRMS calcd for C₁₄H₂₀N₂O₃: 264.1474, found 264.1480.

### Example 7

### (2,6-Dimethylpyridin-4-yl)methyl morpholine-4-carboxylate hydrochloride

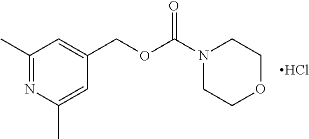

To a solution of (2,6-dimethylpyridin-4-yl)methyl 4-nitrophenyl carbonate (Intermediate 2; 440 mg, 1.46 mmol) in DMF (6 mL) was added DIPEA (320 μL, 2.3 mmol), morpholine (130 μL, 1.5 mmol) and DMAP (10 mg, cat.). The reaction mixture was stirred for five days and then concentrated in vacuo. The residue was purified by normal phase chromatography (gradient eluting with MeOH in DCM from 0% to 10%) followed by reverse phase chromatography to give a colourless oil. The oil was dissolved in Et₂O (5 mL), treated with 2M HCl in Et₂O (1.0 mL, 2.0 mmol) and concentrated in vacuo to give (2,6-dimethylpyridin-4-yl)methyl morpholine-4-carboxylate hydrochloride (226 mg, 54%) as a white solid.

Analytical HPLC: purity 99.7% (System B, R_{T}=3.44 min); Analytical LCMS: purity 100% (System D, R_{T}=4.18 min), ES⁺: 251.3 [MH]⁺; HRMS calcd for C₁₃H₁₈N₂O₃: 250.1317, found 250.1327.

### Example 8

### (2,6-Dimethylpyridin-4-yl)methyl (2R,6S)-2,6-dimethylmorpholine-4-carboxylate hydrochloride

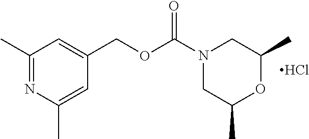

To a solution of (2,6-dimethylpyridin-4-yl)methyl 4-nitrophenyl carbonate (Intermediate 2; 572 mg, 1.89 mmol) in DMF (15 mL) was added cis-2,6-dimethylmorpholine (250 μL, 2.0 mmol), NEt₃ (400 μL, 2.9 mmol) and DMAP (10 mg, cat.). The reaction mixture was stirred overnight and then concentrated in vacuo. The residue was dissolved in EtOAc (50 mL), washed with 1M aq Na₂CO₃ solution (3×50 mL), dried (MgSO₄) and concentrated in vacuo. The crude product was purified by normal phase chromatography (gradient eluting with MeOH in DCM from 0 to 5%) followed by reverse phase chromatography (gradient eluting with MeOH in water, with 1% formic acid in each solvent) to give a colourless gum. The gum was dissolved in DCM (5 mL), treated with 2M HCl in Et₂O (1 mL) and concentrated in vacuo to give (2,6-dimethylpyridin-4-yl)methyl (2R,6S)-2,6-dimethylmorpholine-4-carboxylate hydrochloride (301 mg, 51%) as a white powder.

Analytical HPLC: purity 99.6% (System B, R_{T}=3.79 min); Analytical LCMS: purity 100% (System D, R_{T}=5.21 min), ES⁺: 279.4 [MH]⁺; HRMS calcd for C₁₅H₂₂N₂O₃: 278.1630, found 278.1641.

### Example 9

### (2,6-Dimethylpyridin-4-yl)methyl piperazine-1-carboxylate dihydrochloride

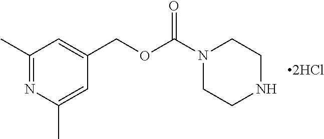

tert-Butyl piperazine-1-carboxylate (5.58 g, 30 mmol), (2,6-dimethylpyridin-4-yl)methyl 4-nitrophenyl carbonate (Intermediate 2; 9.06 g, 30 mmol) and DIPEA (10.4 mL, 60 mmol) were dissolved in DMF (50 mL). The reaction mixture was stirred at r.t. for 16 hours and then concentrated in vacuo. The residue was dissolved in EtOAc (200 mL), washed with aq 1M Na₂CO₃ solution and concentrated in vacuo. The residue was purified by reverse phase chromatography (gradient eluting with MeOH in water from 10% to 100%, with 1% formic acid in each solvent). The residue was dissolved in DCM (50 mL) and excess 2M HCl in Et₂O was added. The mixture was stirred for 16 hours, the precipitate collected by filtration and dried in vacuo to give (2,6-dimethylpyridin-4-yl)methyl piperazine-1-carboxylate dihydrochloride (7.04 g, 73%) as a colourless solid.

Analytical HPLC: purity 100% (System C, R_{T}=6.35 min); Analytical LCMS: purity 100% (System E, R_{T}=6.29 min), ES⁺: 250.4[MH]⁺; HRMS calcd for C₁₃H₁₉N₃O₂: 249.1477, found 249.1484.

### Example 10

### (2,6-Dimethylpyridin-4-yl)methyl 4-ethylpiperazine-1-carboxylate dihydrochloride

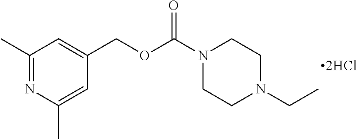

To a solution of (2,6-dimethylpyridin-4-yl)methyl 4-nitrophenyl carbonate (Intermediate 2; 9.07 g, 30 mmol) in DMF (100 mL) was added 1-ethylpiperazine (3.43 g, 30 mmol), triethylamine (3.03 g, 30 mmol) and DMAP (10 mg, cat.). The reaction mixture was stirred overnight and then concentrated in vacuo. The residue was taken up in EtOAc (200 mL), washed with sat aq Na₂CO₃ solution (˜8×200 mL), dried (MgSO₄) and concentrated in vacuo. The residue was purified by reverse phase chromatography (gradient eluting with MeOH in water from 0% to 50%). The residue was taken up in DCM, filtered and the filtrate evaporated to dryness in vacuo to give (2,6-dimethylpyridin-4-yl)methyl 4-ethylpiperazine-1-carboxylate (4.83 g, 58%) as a colourless oil.

The dihydrochloride salt was prepared by treating a solution of (2,6-dimethylpyridin-4-yl)methyl 4-ethylpiperazine-1-carboxylate (1.8 g, 6.5 mmol) in DCM with 2M HCl in Et₂O (8 mL, 16 mmol). The solvents were removed in vacuo to give (2,6-dimethylpyridin-4-yl)-methyl 4-ethylpiperazine-1-carboxylate dihydrochloride (2.28 g, 100%) as a white foam.

Analytical HPLC: purity 100% (System B, R_{T}=2.54 min); Analytical LCMS: purity 100% (System F, R_{T}=4.22 min), ES⁺: 278.5 [MH]⁺; HRMS calcd for C₁₅H₂₃N₃O₂: 277.1790, found 277.1800.

### Example 11

### (2,6-Dimethylpyridin-4-yl)methyl (3S)-3-hydroxypiperidine-1-carboxylate

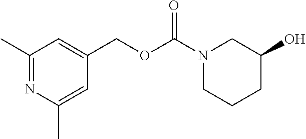

To a stirred solution of (S)-3-hydroxypiperidine hydrochloride (0.50 g, 3.6 mmol) and DIPEA (1.25 mL, 7.2 mmol) in DMF (15 mL) was added DMAP (44 mg, 0.36 mmol) and (2,6-dimethylpyridin-4-yl)methyl 4-nitrophenyl carbonate (Intermediate 2; 1.01 g, 3.6 mmol). The reaction mixture was stirred overnight and then concentrated in vacuo. The residue was purified by reverse phase chromatography (gradient eluting with MeOH in water from 0 to 40%) followed by purification using a 10 g SCX column (eluting with MeOH followed by 1% ammonia in MeOH). The residue was taken up in DCM and treated with charcoal, filtered and dried in vacuo to give (2,6-dimethylpyridin-4-yl)methyl (3S)-3-hydroxypiperidine-1-carboxylate (450 mg, 47%) as a white crystalline solid.

Analytical HPLC: purity 100% (System B, R_{T}=3.11 min); Analytical LCMS: purity 100% (System F, R_{T}=4.74 min), ES⁺: 265.4 [MH]⁺; HRMS calcd for C₁₄H₂₀N₂O₃: 264.1474, found 264.1482.

### Example 12

### (2,6-Dimethylpyridin-4-yl)methyl 4-methylpiperazine-1-carboxylate

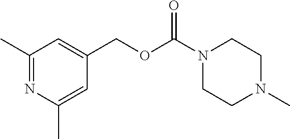

To a solution of (2,6-dimethylpyridin-4-yl)methyl 4-nitrophenyl carbonate (Intermediate 2; 0.604 g, 2.0 mmol) in DMF (10 mL) and added DIPEA (0.520 mL, 3.0 mmol) and N-methylpiperazine (0.222 mL, 3.0 mmol). The reaction mixture was stirred at r.t. overnight and then concentrated in vacuo. The residue was purified by normal phase chromatography (gradient eluting with MeOH in DCM from 0% to 5%) followed by reverse phase chromatography to give (2,6-dimethylpyridin-4-yl)methyl 4-methylpiperazine-1-carboxylate (226 mg, 42%) as a white solid.

Analytical HPLC: purity 100% (System B, R_{T}=2.45 min); Analytical LCMS: purity 97.4% (System F, R_{T}=4.07 min), ES⁺: 264.4 [MH]⁺; HRMS calcd for C₁₄H₂₁N₃O₂: 263.1634, found 263.1647.

### Example 13

### (2,6-Dimethylpyridin-4-yl)methyl (2S)-2,4-dimethylpiperazine-1-carboxylate dihydrochloride

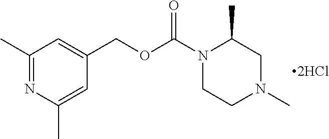

To a solution of (2,6-dimethylpyridin-4-yl)methyl 4-nitrophenyl carbonate (Intermediate 2; 870 mg, 2.89 mmol) in DMF (20 mL) was added NEt₃ (0.80 mL, 5.70 mmol) and (S)-tert-butyl 3-methylpiperazine-1-carboxylate (670 mg, 3.35 mmol). The reaction mixture was stirred at r.t. for 13 days and then concentrated in vacuo. The residue was dissolved in EtOAc (50 mL), washed with 1M aq Na₂CO₃ solution (5×50 mL), dried (MgSO₄) and concentrated in vacuo. The residue was purified by normal phase chromatography (gradient eluting with MeOH in DCM from 0% to 2%) to give (S)-4-tert-butyl 1-(2,6-dimethylpyridin-4-yl)methyl 2-methylpiperazine-1,4-dicarboxylate as a yellow oil. This was dissolved in a mixture of TFA (10 mL) and DCM (15 mL), stirred for 5 hours and then concentrated in vacuo to give (S)-(2,6-dimethylpyridin-4-yl)methyl 2-methylpiperazine-1-carboxylate bistrifluoroacetic acid (1.43 g, 100%) as a light brown oil. A portion of (S)-(2,6-dimethylpyridin-4-yl)methyl 2-methylpiperazine-1-carboxylate bis-trifluoroacetic acid (347 mg, 0.71 mmol) was dissolved in a 1:1 mixture of formic acid and 37% formaldehyde in water (15 mL) and refluxed for 2 hours. The reaction mixture was cooled to ambient, left to stand over the weekend and then carefully poured into 1M aq Na₂CO₃ solution (100 mL). The resulting solution was basified to pH 12 with solid KOH and extracted with EtOAc (3×100 mL). The combined organic layers were dried (MgSO₄) and concentrated in vacuo. The residue was purified by reverse phase chromatography (gradient eluting with MeOH in water from 0% to 100%, with 1% formic acid in each solvent) to give a colourless oil. The oil was dissolved in DCM, treated with an excess of 2M HCl in Et₂O and dried in vacuo to give (2,6-dimethylpyridin-4-yl)methyl (2S)-2,4-dimethylpiperazine-1-carboxylate dihydrochloride (82 mg, 33%) as a white powder.

Analytical HPLC: purity 99.8% (System B, R_{T}=2.57 min); Analytical LCMS: purity 97.4% (System F, R_{T}=4.25 min), ES⁺: 278.5 [MH]⁺; HRMS calcd for C₁₅H₂₃N₃O₂: 277.1790, found 277.1802.

### Example 14

### (2,6-Dimethylpyridin-4-yl)methyl 4-acetylpiperazine-1-carboxylate hydrochloride

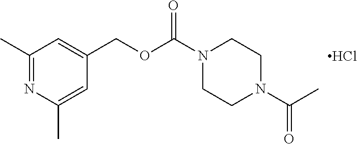

To a solution of (2,6-dimethylpyridin-4-yl)methyl 4-nitrophenyl carbonate (Intermediate 2; 1.05 g, 3.5 mmol) in DMF (25 mL) was added triethylamine (0.60 mL, 4.3 mmol) and 1-acetylpiperazine (0.56 g, 4.4 mmol). The reaction mixture was stirred at r.t. for 8 days and then concentrated in vacuo. The residue was dissolved in EtOAc (50 mL), washed with 1M aq Na₂CO₃ solution (5×50 mL), dried (MgSO₄) and concentrated in vacuo. The crude product was purified by normal phase chromatography (gradient eluting with MeOH in DCM from 0% to 5%). The residue was dissolved in DCM (10 mL), treated with 2M HCl in Et₂O (2.0 mL, 4.0 mmol) and dried in vacuo to give (2,6-dimethylpyridin-4-yl)methyl 4-acetylpiperazine-1-carboxylate hydrochloride (212 mg, 38%) as a white solid.

Analytical HPLC: purity 98.1% (System B, R_{T}=2.96 min); Analytical LCMS: purity 100% (System F, R_{T}=4.72 min), ES⁺: 292.5 [MH]⁺; HRMS calcd for C₁₅H₂₁N₃O₃: 291.1583, found 291.1590.

### Example 15

### Pyridin-3-ylmethyl (2R,6S)-2,6-dimethylmorpholine-4-carboxylate hydrochloride

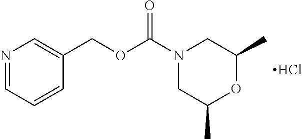

To a solution of 4-nitrophenyl (pyridin-3-yl)methyl carbonate (Intermediate 3; 350 mg, 1.28 mmol) in DMF (10 mL) was added DIPEA (0.22 mL, 1.28 mmol), DMAP (10 mg) and cis-2,6-dimethylmorpholine (0.16 mL, 1.28 mmol). The reaction mixture was stirred at r.t. for 17 hours and then concentrated in vacuo. The residue was dissolved in EtOAc (15 mL), washed with 1M aq NaHCO₃ solution, dried (MgSO₄) and concentrated in vacuo. The residue was purified by normal phase chromatography (gradient eluting with MeOH in DCM from 0% to 2%) followed by reverse phase chromatography. The colourless oil obtained was dissolved in MeOH (4 mL), treated with 2M HCl in Et₂O (0.5 mL, 1.0 mmol) and dried in vacuo to give pyridin-3-ylmethyl (2R,6S)-2,6-dimethylmorpholine-4-carboxylate hydrochloride (120 mg, 25%) as a white solid.

Analytical HPLC: purity 98.9% (System B, R_{T}=3.42 min); Analytical LCMS: purity 100% (System F, R_{T}=5.04 min), ES⁺: 251.4 [MH]⁺; HRMS calcd for C₁₃H₁₈N₂O₃: 250.1317, found 250.1327.

### Example 16

### (6-Methylpyridin-3-yl)methyl morpholine-4-carboxylate

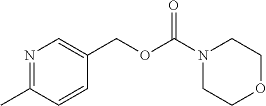

To a solution of (6-methylpyridin-3-yl)methyl 4-nitrophenyl carbonate (Intermediate 4; 864 mg, 3.0 mmol) in DMF (40 mL) was added DIPEA (1.04 mL, 6.0 mmol), DMAP (10 mg, cat.) and morpholine (0.26 mL, 3.0 mmol). The reaction mixture was stirred at r.t. for 2 hours and then concentrated in vacuo. The residue was purified by normal phase chromatography (gradient eluting with MeOH in DCM from 0% to 1%) followed by reverse phase chromatography. The yellow oil obtained was dissolved in EtOAc (70 mL), washed with 1M aq Na₂CO₃ solution, dried (MgSO₄) and concentrated in vacuo. The white crystalline solid obtained was purified by reverse phase chromatography and dried in vacuo to give (6-methylpyridin-3-yl)methyl morpholine-4-carboxylate (185 mg, 26%) as a white solid.

Analytical HPLC: purity 99.4% (System B, R_{T}=2.90 min); Analytical LCMS: purity 100% (System F, R_{T}=4.64 min), ES⁺: 237.4 [MH]⁺; HRMS calcd for C₁₂H₁₆N₂O₃: 236.1161, found 236.1169.

### Example 17

### (6-Methylpyridin-3-yl)methyl 4-ethylpiperazine-1-carboxylate

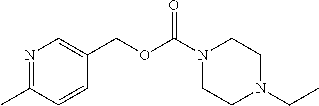

To a solution of (6-methylpyridin-3-yl)methyl 4-nitrophenyl carbonate (Intermediate 4; 576 mg, 2.0 mmol) in DMF (10 mL) was added DIPEA (0.52 mL, 2.0 mmol), DMAP (10 mg, cat.) and N-ethyl piperazine (254 μL, 2.0 mmol). The reaction mixture was stirred at r.t. for 67 hours and then concentrated in vacuo. The crude product was purified by normal phase chromatography (gradient eluting with MeOH in DCM from 0% to 2%) followed by reverse phase chromatography and dried in vacuo to give (6-methylpyridin-3-yl)methyl 4-ethylpiperazine-1-carboxylate (90 mg, 17%) as a yellow oil.

Analytical HPLC: purity 99.1% (System B, R_{T}=2.38 min); Analytical LCMS: purity 100% (System F, R_{T}=3.99 min), ES⁺: 264.4 [MH]⁺; HRMS calcd for C₁₄H₂₁N₃O₂: 263.1634, found 263.1633.

### Example 18

### (2-Methylpyridin-3-yl)methyl morpholine-4-carboxylate

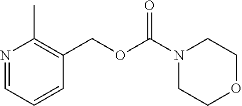

To a solution of (2-methylpyridin-3-yl)methyl 4-nitrophenyl carbonate (Intermediate 5; 1.28 g, 3.0 mmol) in DMF (40 ml) was added DIPEA (1.04 mL, 6.0 mmol), DMAP (10 mg, cat.) and morpholine (0.26 ml, 3.0 mmol). The reaction mixture was stirred at r.t. for 3 hours and then concentrated in vacuo. The crude product was purified by normal phase column chromatography (gradient eluting with MeOH in DCM from 0% to 1%) followed by reverse phase chromatography. The yellow oil obtained was dissolved in EtOAc (70 mL), washed with 1M aq Na₂CO₃ solution, dried (MgSO₄) and concentrated in vacuo to give (2-methylpyridin-3-yl)methyl morpholine-4-carboxylate (413 mg, 58%) as a yellow oil.

Analytical HPLC: purity 100% (System B, R_{T}=2.89 min); Analytical LCMS: purity 100% (System F, R_{T}=4.61 min), ES⁺: 237.4 [MH]⁺; HRMS calcd for C₁₂H₁₆N₂O₃: 236.1161, found 236.1168.

### Example 19

### (6-Methylpyridin-2-yl)methyl morpholine-4-carboxylate hydrochloride

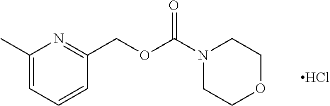

A solution of (6-methylpyridin-2-yl)methanol (369 mg, 3.0 mmol) in THF (10 mL) was added dropwise to a suspension of sodium hydride (60% in mineral oil, 150 mg, 3.75 mmol) in THF (10 mL). The reaction mixture was stirred for 2 minutes and then a solution of 4-morpholinecarbonyl chloride (385 μL, 3.3 mmol) in THF (10 mL) was added drop-wise. The reaction mixture was stirred at r.t. for 2 hours, quenched by the addition of ice and concentrated in vacuo. The residue was dissolved in EtOAc (50 mL), washed with water (20 mL), dried (MgSO₄) and concentrated in vacuo. The residue was purified by reverse phase chromatography. The product was dissolved in DCM, treated with 2M HCl in Et₂O (excess) and dried in vacuo to give (6-methylpyridin-2-yl)methyl morpholine-4-carboxylate hydrochloride (752 mg, 74%) as a pale yellow oil.

is Analytical HPLC: purity 97.0% (System B, R_{T}=2.93 min); Analytical LCMS: purity 99.2% (System F, R_{T}=4.66 min), ES⁺: 237.4 [MH]⁺; HRMS calcd for C₁₂H₁₆N₂O₃: 236.1161, found 236.1165.

### Example 20

### (2,4-Dimethylpyridin-3-yl)methyl morpholine-4-carboxylate

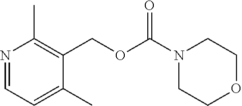

To a solution of (2,4-dimethylpyridin-3-yl)methyl 4-nitrophenyl carbonate (Intermediate 6; 906 mg, 3.0 mmol) in DMF (40 mL) was added DIPEA (1.04 mL, 6.0 mmol), DMAP (10 mg, cat.) and morpholine (0.261 mL, 3.0 mmol). The reaction mixture was stirred at r.t. for 3.5 hours and then concentrated in vacuo. The residue was purified by normal phase chromatography (gradient eluting with MeOH in DCM from 0% to 2%) followed by reverse phase chromatography. The yellow oil obtained was dissolved in EtOAc (70 mL), washed with 1M aq Na₂CO₃ solution, dried (MgSO₄) and concentrated in vacuo to give morpholine-4-carboxylic acid (2,4-dimethylpyridin-3-yl)methyl morpholine-4-carboxylate (277 mg, 37%) as a white solid.

Analytical HPLC: purity 99.8% (System B, R_{T}=3.09 min); Analytical LCMS: purity 100% (System F, R_{T}=4.83 min), ES⁺: 251.4 [MH]⁺; HRMS calcd for C₁₃H₁₈N₂O₃: 250.1317, found 250.1326.

### Example 21

### N-Ethyl-N-(pyridin-4-ylmethyl)morpholine-4-carboxamide hydrochloride

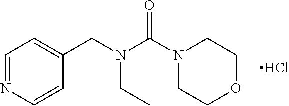

4-Morpholinecarbonyl chloride (8.17 ml, 70 mmol) was added to a suspension of PS-DMAP resin (21.5 g, 1.63 mmol/g, 35 mmol, Argonaut) in DCM (100 mL) and the reaction mixture was shaken for 4 hours at r.t. The resin was filtered and washed with DCM (5×100 mL). The resin was suspended in DCM (150 mL) and 4-(ethylaminomethyl)pyridine (4.77 g, 35 mmol) was added. The reaction mixture was shaken overnight at r.t. The resin was filtered and washed with DCM (5×200 mL). The combined filtrates were concentrated in vacuo. The residue was purified by normal phase chromatography (gradient eluting with MeOH in DCM from 0% to 2%). The residue was dissolved in water (20 mL), treated with charcoal, filtered and dried in vacuo to give N-ethyl-N-(pyridin-4-ylmethyl)morpholine-4-carboxamide hydrochloride (640 mg, 7%) as a colourless liquid.

Analytical HPLC: purity 99.7% (System B, R_{T}=2.93 min); Analytical LCMS: purity 100% (System D, R_{T}=4.68 min), ES⁺: 250.4 [MH]⁺; HRMS calcd for C₁₃H₁₉N₃O₂: 249.1477, found 249.1480

### Example 22

### N-[(2,6-Dimethylpyridin-4-yl)methyl]morpholine-4-carboxamide hydrochloride

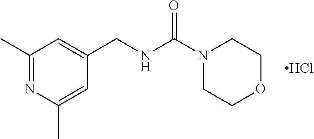

To a suspension of (2,6-dimethylpyridin-4-yl)methanamine dihydrochloride (Intermediate 7; 418 mg, 2.0 mmol) in DMF was added DIPEA (696 μL, 6.0 mmol) and 4-morpholine carbonyl chloride (230 μL, 2.0 mmol). The mixture was stirred for 16 hours at r.t. and then concentrated in vacuo. The residue was purified by normal phase chromatography (gradient eluting with MeOH in DCM from 0% to 5%) followed by preparative HPLC (eluting with water). The residue was dissolved in DCM (1.5 mL), treated with 2M HCl in Et₂O (few drops) and dried in vacuo to give N-[(2,6-dimethylpyridin-4-yl)methyl]-morpholine-4-carboxamide hydrochloride (63 mg, 11%) as a white crystalline solid.

Analytical HPLC: purity 100% (System B, R_{T}=2.63 min); Analytical LCMS: purity 100% (System F, R_{T}=4.42 min), ES⁺: 250.4 [MH]⁺; HRMS calcd for C₁₃H₁₉N₃O₂: 249.1477, found 249.1482.

### Example 23

### N-[(2,6-Dimethylpyridin-4-yl)methyl]-N-ethylmorpholine-4-carboxamide

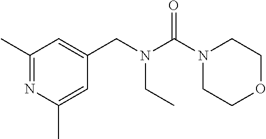

To a solution of (4-methyl-N-ethylamine)-2,6 dimethylpyridine (Intermediate 8; 679 mg, 4.13 mmol) and DIPEA (1.44 mL, 8.27 mmol) in DMF (30 mL) was added 4-morpholine carbonyl chloride (475 μL, 4.13 mmol). The mixture was stirred for 72 hours at r.t. and then concentrated in vacuo. The residue was dissolved in DCM (100 mL), washed with sat aq NaHCO₃ solution (3×25 mL), dried (MgSO₄) and concentrated in vacuo. The residue was purified by reverse phase chromatography (eluting with MeOH in water from 0% to 100%, with 1% formic acid in each solvent). The product was dissolved in EtOAc (100 mL), washed with sat aq NaHCO₃ solution (25 mL), dried (MgSO₄) and concentrated in vacuo to give N-[(2,6-dimethylpyridin-4-yl)methyl]-N-ethylmorpholine-4-carboxamide (48 mg, 4%) as a colourless oil.

Analytical HPLC: purity 99.3% (System B, R_{T}=4.12 min); Analytical LCMS: purity 98% (System D, R_{T}=6.13 min), ES⁺: 278.5 [MH]⁺.

### Biological Tests

Measurement of Overnight Body Weight Change in Male C57 bl/6 Mice

This model studies the effects of compounds on body weight gain during the pm-am period in order to maximise the effective window. Typically the mice gain about 1 g in weight during the dark phase and then loose the majority of this weight gain during the light phase, as represented in FIG. 1. The weight difference over any 24 hour period is very small whilst the weight difference between the beginning of the dark phase and the beginning of the light phase (pm-am) is maximal.

It is important to measure body weight change over the dark phase. If mice are dosed with an active compound on two consecutive days and the bodyweight change is recorded 48 hours after the first dose then no significant effect is observed. However if the body weight is change over the dark phase only is considered a significant and robust effect is seen. This is because the mice rebound during the light phase to compensate for the lack of weight gain over the dark phase. Very active long lasting compounds may also diminish this rebound and reduce the body weight over the 48 hours.

Weight Change Over Consecutive Days in C57bl\6 male mice:

The weight difference between the beginning of the dark phase and the beginning of the light phase (pm-am) is greater than the weight difference measured between pm and pm on 2 consecutive days. The effect of the compounds on the pm-am difference was therefore studied in order to maximise the effect window.

C57 bl/6 mice were grouped (5 per cage) and left 5 days for acclimatization. A single intraperitoneally (ip) administered dose (60 mg/kg) was given just prior to the dark phase. Compounds were either water soluble or dissolved in up to 3% cremophor (in this case the vehicle also contained cremophor). The pH was adjusted from a minimum of 5.5 to a maximum of 8 depending on the nature of the compound.

As shown in FIGS. 2 to 5, compounds of Formula (I) are useful for decreasing body weight in mice.

### Leptin Assay in Non-Recombinant System

Although well-characterised in recombinant systems (e.g. ObRb-transfected HEK293 cells), where leptin elicits a very marked increase in STAT3 phosphorylation, these systems have often failed to provide an accurate measure of activity of a test compound towards the leptin receptor. It seems that overexpression of the receptor (as well as the possibility for different drugs to act on different parts of the signaling pathway triggered by leptin association with its receptor) results in most cases in the absence of activity of the drugs tested.

The leptin receptor expression in non-recombinant system is often fluctuating and care must be given to identify a system where signal stability remains within experiments. Using such a system, leptin receptor antagonist mimetics could be identified by evaluating is their action vs. leptin (see below).

Leptin is produced chiefly in adipose cells, but in humans, mRNA encoding leptin is also present in the placenta. Here, leptin might play an important proliferative role in the microvasculature. The possibility to use this hypothesis in a native cell line was evaluated.

### JEG-3 Protocol

In JEG-3 cells (choriocarcinoma cell line) leptin is able to stimulate proliferation up to 3 fold (Biol. Reprod. (2007) 76: 203-10). Leptin also causes a concentration-dependent increase in [³H]-thymidine incorporation in JEG-3 cells (FIG. 6, maximal effect at 100 nM (EC₅₀=2.1 nM)). The radioactivity incorporated by the cells is an index of their proliferative activity and is measured in counts per minute (CPM) with a liquid scintillation beta counter.

This finding can be applied to test whether a compound is able to either reproduce the effect of leptin on cell proliferation (leptin receptor agonist mimetic) (i.e., a given compound will cause an increase in incorporated [³H]-Thymidine by the cells) or to inhibit the effect of leptin (antagonistic effect) by preventing the leptin-mediated increase in [³H]-thymidine incorporation.

This approach has the advantage of using a non-recombinant system and has reasonable reproducibility and robustness.

### Measurement of Brain Penetration

The test species (rodent) is given a bolus dose of the substrate under investigation, usually via intravenous (IV) or oral (PO) routes. At appropriate time points, blood samples are taken and the resultant plasma extracted and analysed for substrate concentration and, where appropriate, metabolite concentration. At similar time points, animals from another group are sacrificed, brains isolated and the brain surface cleaned. Brain samples are then homogenised, extracted and analysed for substrate concentration and, where appropriate, metabolite concentration. Alternatively, microdialysis probes are implanted into one or more brain regions of the test species and samples collected at appropriate time points for is subsequent analysis. This method has the advantage of measuring only extra-cellular substrate concentration. Plasma and brain concentrations are then compared and ratios calculated, either by comparison of averaged concentrations at individual time points, or by calculation of the area-under-the-curve (AUC) of the concentration-time plots.

1. A compound of formula (I) 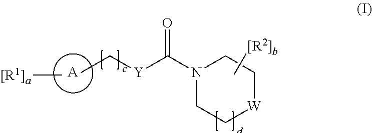
or a pharmaceutically acceptable salt, solvate, hydrate, geometrical isomer, tautomer, optical isomer or N-oxide thereof, wherein:
A is a pyridine ring;
Y is O, N(R³) or CH₂;
W is O, N(R⁴) or CH₂;
each R¹ is independently selected from C₁₋₄-alkyl, C₁₋₄-alkoxy, halogen, cyano and CF₃;
each R² is independently selected from hydroxy and C₁₋₄-alkyl;
R³ is hydrogen or C₁₋₄-alkyl;
R⁴ is selected from hydrogen, C₁₋₆-alkyl, C₁₋₆-acyl, phenyl and benzyl, wherein phenyl and benzyl are optionally substituted with one or more substituents selected from halogen, cyano, CF₃, C₁₋₆-alkyl, C₁₋₆-alkoxy, phenyl and phenoxy;
a and b are each independently 0, 1 or 2;
c is 1 or 2; and
d is 0, 1 or 2;
provided that the compound is not selected from the group consisting of:
N-(4-pyridinylmethyl)-4-morpholinecarboxamide;
4-(3-methylphenyl)-N-(2-pyridinylmethyl)-1-piperazinecarboxamide;
4-(4-fluorophenyl)-N-(3-pyridinylmethyl)-1-piperazinecarboxamide;
N-(2-pyridinylmethyl)-4-morpholinecarboxamide;
4-(2-methylphenyl)-N-(3-pyridinylmethyl)-1-piperazinecarboxamide;
4-(5-chloro-2-methylphenyl)-N-(3-pyridinylmethyl)-1-piperazinecarboxamide;
N-(3-pyridinylmethyl)-4-morpholinecarboxamide;
(2R,5S)-4-[4-cyano-3-(trifluoromethyl)phenyl]-N,2,5-trimethyl-N-(2-pyridinyl-methyl)-1-piperazinecarboxamide;
(2R,5S)-4-[4-cyano-3-(trifluoromethyl)phenyl]-2,5-dimethyl-N-[(3-methyl-2-pyridinyl)methyl]-1-piperazinecarboxamide;
(2-pyridinyl)methyl 4-methylpiperazine-1-carboxylate;
(2R,5S)-4-[4-cyano-3-(trifluoromethyl)phenyl]-2,5-dimethyl-N-(3-pyridinyl-methyl)-1-piperazinecarboxamide;
2-(2-pyridinyl)ethyl 4-methylpiperazine-1-carboxylate;
4-phenyl-N-[2-(2-pyridinyl)ethyl]-1-piperazinecarboxamide;
4-(2,3-dimethylphenyl)-N-(3-pyridinylmethyl)-1-piperazinecarboxamide;
(2R,5S)-4-[4-cyano-3-(trifluoromethyl)phenyl]-2,5-dimethyl-N-[2-(4-pyridinyl)-ethyl]-1-piperazinecarboxamide;
4-(4-chlorophenyl)-N-(3-pyridinylmethyl)-1-piperidinecarboxamide;
N-[2-(4-pyridinyl)ethyl]-4-morpholinecarboxamide;
4-phenyl-N-(3-pyridinylmethyl)-1-piperazinecarboxamide;
(2R,5S)-4-[4-cyano-3-(trifluoromethyl)phenyl]-N,2,5-trimethyl-N-(3-pyridinyl-methyl)-1-piperazinecarboxamide;
4-phenyl-N-(2-pyridinylmethyl)-1-piperazinecarboxamide;
N-[[3-chloro-5-(trifluoromethyl)-2-pyridinyl]methyl]-4-morpholinecarboxamide;
(2R,5S)—N-[(6-chloro-2-pyridinyl)methyl]-4-[4-cyano-3-(trifluoromethyl)phenyl]-2,5-dimethyl-1-piperazinecarboxamide;
(2R,5S)—N-[(6-chloro-3-pyridinyl)methyl]-4-[4-cyano-3-(trifluoromethyl)phenyl]-2,5-dimethyl-1-piperazinecarboxamide;
1-[3-[3-chloro-5-(trifluoromethyl)-2-pyridinyl]-1-oxopropyl]-4-phenyl-piperazine;
1-[3-(2-pyridyl)propionyl]-piperidine;
1-methyl-4-[1-oxo-3-(3-pyridinyl)propyl]-piperazine;
1-[3-[3-chloro-5-(trifluoromethyl)-2-pyridinyl]-1-oxopropyl]-4-methyl-piperazine;
4-[3-[3-chloro-5-(trifluoromethyl)-2-pyridinyl]-1-oxopropyl]-morpholine;
(6-methylpyridin-2-yl)methyl 4-(2,4,6-trimethoxybenzyl)-piperazine-1-carboxylate;
4-(5-fluoro-2-methoxybenzyl)-N-[2-(pyridin-2-yl)ethyl]-1-piperazinecarboxamide;
N-[(6-methoxy-3-pyridinyl)methyl]-4-phenyl-1-piperazinecarboxamide;
4-(4-fluorophenyl)-N-[(6-methoxy-3-pyridinyl)methyl]-1-piperazinecarboxamide;
4-(2-fluorophenyl)-N-[(6-methoxy-3-pyridinyl)methyl]-1-piperazinecarboxamide;
(2R,5S)-4-[4-cyano-3-(trifluoromethyl)phenyl]-N-[(6-methoxy-3-pyridinyl)-methyl]-2,5-dimethyl-1-piperazinecarboxamide;
1-[1-oxo-3-(3-pyridinyl)propyl]-piperazine;
4-[1-oxo-3-(2-pyridinyl)propyl]-morpholine;
4-(3-chlorophenyl)-N-[(6-methoxy-3-pyridinyl)methyl]-1-piperazinecarboxamide;
4-(4-methoxyphenyl)-N-(3-pyridinylmethyl)-1-piperazinecarboxamide; and
N-[(1-oxidopyridin-3-yl)methyl]piperidine-1-carboxamide.

2. A compound according to claim 1, wherein Y is O.

3. A compound according to claim 1, wherein Y is N(R³).

4. A compound according to claim 1, which is selected from:
pyridin-4-ylmethyl morpholine-4-carboxylate;
pyridin-4-ylmethyl (3R)-3-hydroxypyrrolidine-1-carboxylate;
pyridin-4-ylmethyl (2R,6S)-2,6-dimethylmorpholine-4-carboxylate;
pyridin-4-ylmethyl 4-ethylpiperazine-1-carboxylate;
pyridin-4-ylmethyl 4-phenylpiperazine-1-carboxylate;
2-pyridin-4-ylethyl (2R,6S)-2,6-dimethylmorpholine-4-carboxylate;
(2,6-dimethylpyridin-4-yl)methyl morpholine-4-carboxylate;
(2,6-dimethylpyridin-4-yl)methyl (2R,6S)-2,6-dimethylmorpholine-4-carboxylate;
(2,6-dimethylpyridin-4-yl)methyl piperazine-1-carboxylate;
(2,6-dimethylpyridin-4-yl)methyl 4-ethylpiperazine-1-carboxylate;
(2,6-dimethylpyridin-4-yl)methyl (3S)-3-hydroxypiperidine-1-carboxylate;
(2,6-dimethylpyridin-4-yl)methyl 4-methylpiperazine-1-carboxylate;
(2,6-dimethylpyridin-4-yl)methyl (2S)-2,4-dimethylpiperazine-1-carboxylate;
(2,6-dimethylpyridin-4-yl)methyl 4-acetylpiperazine-1-carboxylate;
pyridin-3-ylmethyl (2R,6S)-2,6-dimethylmorpholine-4-carboxylate;
(6-methylpyridin-3-yl)methyl morpholine-4-carboxylate;
(6-methylpyridin-3-yl)methyl 4-ethylpiperazine-1-carboxylate;
(2-methylpyridin-3-yl)methyl morpholine-4-carboxylate;
(6-methylpyridin-2-yl)methyl morpholine-4-carboxylate;
(2,4-dimethylpyridin-3-yl)methyl morpholine-4-carboxylate;
N-ethyl-N-(pyridin-4-ylmethyl)morpholine-4-carboxamide;
N-[(2,6-dimethylpyridin-4-yl)methyl]morpholine-4-carboxamide; and
N-[(2,6-dimethylpyridin-4-yl)methyl]-N-ethylmorpholine-4-carboxamide.

5. A pharmaceutical formulation containing a compound according to any one of claims 1 to 4 as active ingredient, in combination with a pharmaceutically acceptable diluent or carrier.

6. A compound according to any one of claims 1 to 4 for use in therapy.

7. A compound according to any one of claims 1 to 4 for use in the treatment or prevention of conditions or diseases associated with weight gain.

8. The compound according to claim 7, wherein the condition or disease is obesity, type 2 diabetes, lipodystrophy, insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain or macular degeneration.

9. A compound according to any one of claims 1 to 4 for use in the treatment or prevention of severe weight loss, dysmenorrhea, amenorrhea, female infertility or immunodeficiency, or in the treatment of wound healing.

10. A compound according to any one of claims 1 to 4 for use in the treatment or prevention of inflammatory conditions or diseases, low level inflammation associated with obesity and excess plasma leptin, atherosclerosis, macro or micro vascular complications of type 1 or 2 diabetes, retinopathy, nephropathy, autonomic neuropathy, or blood vessel damage caused by ischaemia or atherosclerosis.

11. A compound according to any one of claims 1 to 4 for use in the inhibition of angiogenesis.

12. Use of a compound according to any one of claims 1 to 4 in the manufacture of a medicament for the treatment or prevention of conditions or diseases associated with weight gain.

13. The use according to claim 12, wherein the condition or disease is obesity, type 2 diabetes, lipodystrophy, insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain or macular degeneration.

14. Use of a compound according to any one of claims 1 to 4 in the manufacture of a medicament for the treatment or prevention of severe weight loss, dysmenorrhea, amenorrhea, female infertility or immunodeficiency, or for the treatment of wound healing.

15. Use of a compound according to any one of claims 1 to 4 in the manufacture of a medicament for the treatment or prevention of inflammatory conditions or diseases, low level inflammation associated with obesity and excess plasma leptin, atherosclerosis, macro or micro vascular complications of type 1 or 2 diabetes, retinopathy, nephropathy, autonomic neuropathy, or blood vessel damage caused by ischaemia or atherosclerosis.

16. Use of a compound according to any one of claims 1 to 4 in the manufacture of a medicament for the inhibition of angiogenesis.

17. A method for treatment or prevention of conditions or diseases associated with weight gain, which comprises administering to a mammal, including man, in need of such treatment an effective amount of a compound according to any one of claims 1 to 4.

18. The method according to claim 17, wherein the condition or disease is obesity, type 2 diabetes, lipodystrophy, insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain or macular degeneration.

19. A method for treatment or prevention of severe weight loss, dysmenorrhea, amenorrhea, female infertility or immunodeficiency, or for treatment of wound healing, which comprises administering to a mammal, including man, in need of such treatment an effective amount of a compound according to any one of claims 1 to 4.

20. A method for treatment or prevention of inflammatory conditions or diseases, low level inflammation associated with obesity and excess plasma leptin, atherosclerosis, macro or micro vascular complications of type 1 or 2 diabetes, retinopathy, nephropathy, autonomic neuropathy, or blood vessel damage caused by ischaemia or atherosclerosis, which comprises administering to a mammal, including man, in need of such treatment an effective amount of a compound according to any one of claims 1 to 4.

21. A method for inhibition of angiogenesis, which comprises administering to a mammal, including man, in need of such treatment an effective amount of a compound according to any one of claims 1 to 4.
